(19) United States
(12) Patent Application Publication
(10) Pub. No.: US 2025/0122298 A1
(43) Pub. Date: Apr. 17, 2025

(54) DEIMMUNIZED VNAR DOMAINS AND SCAFFOLDS

(71) Applicant: OSSIANIX, INC., Philadelphia, PA (US)

(72) Inventors: Pawel STOCKI, Hertfordshire (GB); Julia Lynn RUTKOWSKI, Bryn Mawr, PA (US); Mykhaylo DEMYDCHUK, Cambridgeshire (GB); Shu-Fen COKER, Buckinghamshire (GB)

(21) Appl. No.: 18/684,463

(22) PCT Filed: Aug. 17, 2022
(86) PCT No.: PCT/US2022/040616

§ 371 Date: Feb. 16, 2024

Related U.S. Application Data
(63) Continuation-in-part of application No. PCT/US2021/058661, filed on Nov. 9, 2021.
(60) Provisional application No. 63/277,590, filed on Nov. 9, 2021.
(60) Provisional application No. 63/234,210, filed on Aug. 17, 2021.

(51) Int. Cl. C07K 16/28

(52) U.S. Cl. CPC C07K 16/2881; C07K 2317/24; C07K 2317/52; C07K 2317/569; C07K 2317/92

(57) ABSTRACT

The present disclosure relates to deimmunized VNAR scaffolds, methods of making the scaffolds and their use as frameworks, and VNAR domains comprising those scaffolds, especially therapeutic VNAR domains to a target antigen of interest, including, for example, high affinity VNAR domains that are cross reactive with primate transferrin receptors (“TfR”) as well as other VNAR domains capable of carrying a therapeutic or diagnostic cargo across the blood brain barrier.

### CROSS REFERENCE TO RELATED APPLICATION

This application is a national phase filing under 35 U.S. § 371 of Intl. Appln. No. PCT/US2022/040616, filed Aug. 17, 2022 which claims the priority and benefit of Intl. Appln. No. PCT/US2021/058661, filed Nov. 9, 2021, provisional application U.S. Ser. No. 63/277,590, filed on Nov. 9, 2021, and provisional application U.S. Ser. No. 63/234,210, filed on Aug. 17, 2021, each of which is incorporated herein by reference in their entirety.

### SEQUENCE LISTING

The instant application contains a Sequence Listing which has been submitted electronically in XML format and is hereby incorporated by reference in its entirety. Said XML copy, created on Feb. 16, 2024 is named 90302002-us4 Sequence Listing,xml and is 21,566 bytes in size.

### FIELD OF THE DISCLOSURE

The present disclosure relates to deimmunized VNAR scaffolds, methods of making the scaffolds and their use as frameworks, and VNAR domains comprising those scaffolds, especially therapeutic VNAR domains to a target antigen of interest, including, for example, high affinity VNAR domains that are cross reactive with primate transferrin receptors (“TfR”) as well as other VNAR domains capable of carrying a therapeutic or diagnostic cargo across the blood brain barrier.

### BACKGROUND

The therapeutic applications of the variable domain of Ig new antigen receptor (VNAR) discovered in sharks are being explored to exploit its unique structure and physiochemical properties [Könning, 2017]. To be therapeutically useful, however, all novel non-human antibodies should be humanized to reduce the risk of immunogenicity. Typically, CDRs from a foreign antibody are grafted onto a human germ line framework, and then back-mutations are introduced at key positions to restore optimal antigen binding [Safdari 2013]. This approach has yielded many humanized antibodies that have successfully been developed into therapeutic products.

Shark VNARs represent a challenge for this type of humanization because of the structural differences (lack of CDR2) and low overall sequence identity (approximately 30%) to corresponding human V domains. Crystal structures of VNAR domains show that the framework organization is similar to some human VL domains [Stanfield 2007], and humanization of a spiny dog fish VNAR was carried out by converting over 60% of the framework residues to those of the germ line human VL domain DPK9 [Kovalenko 2013]. However, the extent of replacement in the VNAR scaffold leads to a dramatic loss in target binding that requires extensive paratope re-engineering that may only be partially successful.

The present disclosure addresses the need for deimmunized VNAR scaffolds. In the present approach to reduce the risk associated with therapeutic VNARs, human T-cell epitopes [De Groot 2005] have been identified in the VNAR scaffold and removed. This approach pairs identification of potential T-cell epitopes and the substitution of key amino acids to abrogate to MHC class II (MHCII) binding using in silico analyses, followed by the synthesis and functional evaluation of the modified proteins using in vitro assays. This approach provides the foundation for the deimmunization of shark VNAR scaffolds and opens up their potential for use in humans.

### SUMMARY OF THE DISCLOSURE

The present disclosure relates to deimmunized Type II VNAR scaffolds, VNAR domains with those scaffolds and methods of identifying deimmunized scaffolds.

Accordingly, one aspect disclosed herein disclosure provides naturally occurring or artificial Type II VNAR scaffolds which contain MHC Class II binding sites which have been deimmunized by independently mutating any one, two or three, or any combination thereof, of the regions with those sites by substitution, deletion, or insertion of from one to five amino acids per region. In an exemplary embodiment, (i) region 1 is amino acid residues 10 to 18 of SEQ ID NO. 10 (i.e., residues ITKETGESL), (ii) region 2 is amino acid residues 55 to 64 of SEQ ID NO. 10 (i.e., residues YVETVNSGSK); and (iii) region 3 is amino acid residues 97 to 106 of SEQ ID NO. 10 (i.e., residues YGGGTAVTVN), when CDR1 is 8 amino acid residues and CDR3 is 12 amino acid residues in length, wherein SEQ ID NO: 10 is shown below:

¹ARVDQTPQTITKETGESLTINCVLR-CDR1-³⁴TYWYRKKSGSTNEEN
ISKGGRYVETVNSGSKSFSLKINDLTVEDSGTYRCN-CDR3-⁹⁷YGGGT
AVTVNA.

The above residue numbers for regions 1, 2 and 3 also correspond exactly with the amino acid residues of SEQ ID NO: 7.

In some embodiments, the Type II VNAR scaffold is for a VNAR domain having a formula of, from N to C terminus,
FW1-CDR1-FW2-HV2-FW2′-HV4-FW3-CDR3-FW4
   Such VNAR scaffolds comprise an amino acid sequence in which

FW1 is
(SEQ ID NO: 4)
ARVDQTPQTITKETGESLTINCVLR;
HV2--FW2'--HV4--FW3 is
(SEQ ID NO: 5)
TYWYRKKSGSTNEENISKGGRYVETVNSGSKS
FSLKINDLTVEDSGTYRCN;
and
FW4 is
(SEQ ID NO: 6)
YGGGTAVTVNA,

wherein CDR1 and CDR3 taken together each have at least one cysteine residue; and
wherein at least one of regions 1, 2 or 3 has one, two or three amino acids changes to reduce or remove the immunogenicity of an MHC class II binding site,
wherein
   (i) region 1 is amino acid residues 10 to 18 of SEQ ID NO: 7 (i.e., residues ITKETGESL),
   (ii) region 2 is amino acid residues 55 to 64 of SEQ ID NO: 7 (i.e., residues YVETVNSGSK); and
   (iii) region 3 is amino acid residues 97 to 106 of SEQ ID NO: 7 (i.e., residues YGGGTAVTVN).

In some embodiments, these VNAR scaffolds are used for VNAR domain which have a CDR1 region with an amino acid sequence of DSNCALSS (SEQ ID NO: 8) in combination with any Type II CDR3 region. In some embodiments, the VNAR domain has CDR1 and CDR3 regions from the same Type II CDR1/CDR3 cognate pair for a target of interest.

In another aspect, this disclosure relates to Type II VNAR domains comprising, from N to C terminus, an amino acid sequence and formula of

(SEQ ID NO: 10)
¹ARVDQTPQTITKETGESLTINCVLR-CDR1-³⁴TYWYRKKSG
STNEENISKGGRYVETVNSGSKSFSLKINDLTVEDSGTYRC
N-CDR3-⁹⁷YGGGTAVTVNA,

wherein CDR1 and CDR3 are a Type II cognate pair for a target antigen of interest; and
wherein at least one of regions 1, 2 or 3 has one, two or three amino acids changes to reduce or remove the immunogenicity of an MHC class II binding site, wherein
   (i) region 1 is amino acid residues 10 to 18 of SEQ ID NO. 7 (i.e., residues ITKETGESL),
   (ii) region 2 is amino acid residues 55 to 64 of SEQ ID NO. 7 (i.e., residues YVETVNSGSK); and
   (iii) region 3 is amino acid residues 97 to 106 of SEQ ID NO. 7 (i.e., residues YGGGTAVTVN).

In embodiments, the amino acid changes for the VNAR scaffolds and domains can include any one of the following mutations, which can be taken independently or in combination,
in region 1, the changes of 110A, 110E, 110S, or I10T,
in region 2, the changes of (a) a one amino acid change of Y55H, Y55N, Y55S or Y55T, or (b) a two amino acid change of N60D and S63T, R54K and N60E, or R54K and N60Q,
in region 3, the changes of (a) a one amino acid change of Y97S, G99D or A102V, or (b) a three amino acid change of Y97S, G99D and A102V,
changes in which at least one amino acid is changed in each of region 1, 2 and 3, wherein any of the foregoing are included as well as amino acid changes are selected from the group consisting of 110A, N60D, S63T, Y97S, G99D and A102V; 110A, R54K, N60E, Y97S, G99D and A102V; 110A, R54K, N60Q, Y97S, G99D and A102V; 110A, Y55H, Y97S, G99D and A102V; 110A, Y55N, Y97S, G99D and A102V; 110A, Y55S, Y97S, G99D and A102V; 110A, Y55T, Y97S, G99D and A102V; I10S, Y55H, Y97S, G99D and A102V; and I10T, Y55H, Y97S, G99D and A102V.

In some embodiments, any of the VNAR domains of the disclosure can comprise the acid sequence between CDR1 and CDR3 of

(SEQ ID NO: 13)
TYWYRKKSGSTNEENISKGGRYVETV
DSGTKSFSLKINDLTVEDSGTYRCN
or
(SEQ ID NO: 14)
TYWYRKKSGSTNEENISKGGRKYVET
VESGSKSFSLKINDLTVEDSGTYRCN.

In some embodiments the VNAR scaffold comprises SEQ ID NO: 15 or 17.

In some embodiments, the VNAR domain has CDR1 and CDR3 regions from the same Type II CDR1/CDR3 cognate pair for a target of interest. In some embodiments, the target of interest is a BBB-shuttling VNAR domain, such as TfR or CD98, a B-lymphocyte stimulator (BAFF), or a COV2 spike protein.

The foregoing embodiments are contemplated to be present in all possible permutations.

In other aspects, the deimmunized VNAR domains are conjugates with a heterologous agent, are immunoconjugates, including but not limited to VNAR antibodies. The disclosure further provides nucleic acids encoding the deimmunized VNAR scaffolds, VNAR domains or conjugates, as well as vectors and host cells containing those nucleic acids and vectors.

Yet further aspects relate to methods of deimmunizing a VNAR scaffold. In an embodiment, the method comprises introducing one or more a deimmunizing mutations at an MHC Class II binding site in regions 1, 2 or 3 or any combination thereof in a naturally occurring or artificial VNAR scaffold comprising one or more of said regions, wherein
(i) region 1 comprises of amino acid residues ITKETGESL (SEQ ID NO: 1),
(ii) (ii) region 2 comprises of amino acid residues YVETVNSGSK (SEQ ID NO: 2) and
(iii) (iii) region 3 comprises of is amino acid residues YGGGTAVTVN (SEQ ID NO: 3).

In some embodiments, the Type II VNAR scaffold is for a VNAR domain having a formula of, from N to C terminus of
FW1-CDR1-FW2-HV2-FW2′-HV4-FW3-CDR3-FW4
   and the scaffold comprises an amino acid sequence wherein

FW1 is
(SEQ ID NO: 4)
ARVDQTPQTITKETGESLTINCVLR;
HV2--FW2'--HV4--FW3 is
(SEQ ID NO: 5)
TYWYRKKSGSTNEENISKGGRYVETVN
SGSKSFSLKINDLTVEDSGTYRCN;
and
FW4 is
(SEQ ID NO. 6)
YGGGTAVTVNA

### BRIEF DESCRIPTION OF THE DRAWINGS

FIG. 1. Cross-species reactivity of TXP1 assessed by ELISA. Plates were coated with either mouse, human, cynomolgus or rat TfR-1. HSA was used as a negative control.

FIG. 2. Examples of VNAR-Antibody Fusion Proteins. Antibodies with a monovalent VNAR (top row) or bivalent VNARs (bottom row) genetically fused to a monoclonal antibody via glycine linkers.

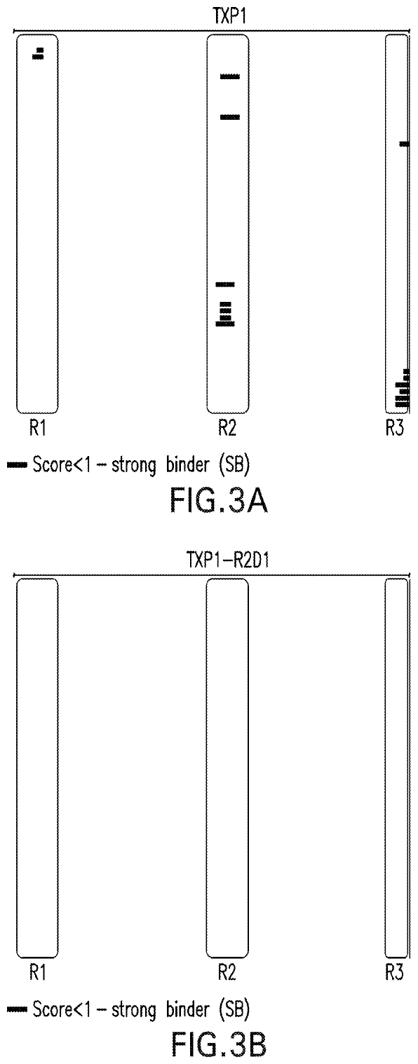

FIGS. 3A-C. Predicted HLA binding for TXP1 deimmunized variants. Scores of less than 1 are strong binders as shown in black for TXP1 VNAR domain (A) but do not appear for the deimmunized variants (B and C).

FIG. 4A-D. Binding activity of TXP1 and deimmunized variants assessed by ELISA. Plates were coated with either human (A) or cynomolgus (B) or rat TfR-1 (C). HSA was used as a negative control. Serial dilutions of TXP1 (a VNAR antibody) or the variants as Fc fusions were added to the plate and binding was measured by end-point ELISA upon incubation with secondary anti-hFc HRP-conjugated antibodies and visualization with TMB. Absorbance was measured at 450 nm and 4-parametric non-linear regression was used to calculate EC50 values (D).

FIG. 5. Identification of 15-mer peptides derived from TXP1 with strong MHCII binding. The 53 most common MHCII alleles were analyzed for binding motifs using NetMHCIIpan-4.0. Each block represents a 15-mer peptide starting from position 1 to 93 to cover the whole TXP1 sequence. Black blocks represented 15-mer peptides with a score <1 (strong binder); 3 regions are marked for deimmunization.

FIG. 6. Expression level and hTfR1 binding of 20 single-region variants of TXP1 measured by ELISA. Variants were designed to remove potential MHCII binding motifs in each region separately with the mutations described in Table 5. Each deimmunized variant was transiently expressed in 0.5 ml scale in ExpiHEK cells and the crude media were tested by ELISA for hTfRIbinding and expression level in comparison to parental TXP1. The data were normalized to TXP1 and the variants with binding or expression >0.5-fold were evaluated further.

FIG. 7. Expression level and hTfR1 binding of 35 fully deimmunized variants of TXP1 measured by ELISA. Variants with mutations in the three immunogenic regions combined (Table 5) were transiently expressed at the 0.5 ml scale in ExpiHEK cells and the crude media were tested by ELISA for hTfR1 binding and expression level in comparison to parental TXP1. The data were normalized to TXP1 and the variants with binding or expression >0.5-fold were evaluated further.

FIG. 8. Analytical SEC of TXP1 variants containing a single deimmunized region. Mutations were introduced into each region separately (Table 5). SEC analysis was performed after protein A purification and the % purity was calculated based the AUC of the monomer peak.

FIG. 9A-C. Internalization of TXP1, TXP1D1 and TXP1D4 by hCMEC/D3 or SH-SY5Y cells. Cells were grown on the collagen coated chamber slides for 48 hr before incubation with VNAR antibodies at 44.3 nM for 1 h at 37° C. Cells were washed, fixed and permeabilized before being incubated with secondary detection antibodies conjugated to Alexa555. Cells were imaged at 20× on fluorescent microscope (A). Data from 3 eye-fields were quantified and presented as corrected total cell fluorescence (CFCF), ±SD (B, hCMEC/D3 cells; C, SH-SY5Y cells).

### DETAILED DESCRIPTION OF THE DISCLOSURE

In order that the present disclosure may be more readily understood, certain terms are defined below. Additional definitions may be found within the detailed description of the disclosure.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The singular forms “a,” “an,” and “the” include the plurals unless the context clearly dictates otherwise.

The term “including” is used to mean “including but not limited to.” “Including” and “including but not limited to” are used interchangeably.

The terms “patient,” “subject,” and “individual” may be used interchangeably and refer to either a human or a non-human animal. These terms include mammals such as humans, primates, livestock animals (e.g., cows, pigs), companion animals (e.g., dogs, cats) and rodents (e.g., mice and rats).

The term “non-human mammal” means a mammal which is not a human and includes, but is not limited to, a mouse, rat, rabbit, pig, cow, sheep, goat, dog, primate, or other non-human mammals typically used in research. As used herein, “mammals” includes the foregoing non-human mammals and humans.

As used herein, “treating” or “treatment” and grammatical variants thereof refer to an approach for obtaining beneficial or desired clinical results. The term may refer to slowing the onset or rate of development of a condition, disorder or disease, reducing or alleviating symptoms associated with it, generating a complete or partial regression of the condition, or some combination of any of the above. For the purposes of this disclosure, beneficial or desired clinical results include, but are not limited to, reduction or alleviation of symptoms, diminishment of extent of disease, stabilization (i.e., not worsening) of state of disease, delay or slowing of disease progression, amelioration or palliation of the disease state, and remission (whether partial or total), whether detectable or undetectable. “Treatment” can also mean prolonging survival relative to expected survival time if not receiving treatment. A subject (e.g., a human) in need of treatment may thus be a subject already afflicted with the disease or disorder in question. The term “treatment” includes inhibition or reduction of an increase in severity of a pathological state or symptoms relative to the absence of treatment and is not necessarily meant to imply complete cessation of the relevant disease, disorder or condition.

As used herein, the terms “preventing” and grammatical variants thereof refer to an approach for preventing the development of, or altering the pathology of, a condition, disease or disorder. Accordingly, “prevention” may refer to prophylactic or preventive measures. For the purposes of this disclosure, beneficial or desired clinical results include, but are not limited to, prevention or slowing of symptoms, progression or development of a disease, whether detectable or undetectable. A subject (e.g., a human) in need of prevention may thus be a subject not yet afflicted with the disease or disorder in question. The term “prevention” includes slowing the onset of disease relative to the absence of treatment and is not necessarily meant to imply permanent prevention of the relevant disease, disorder or condition. Thus “preventing” or “prevention” of a condition may in certain contexts refer to reducing the risk of developing the condition or preventing or delaying the development of symptoms associated with the condition.

As used herein, an “effective amount,” “therapeutically-effective amount” or “effective dose” is an amount of a composition (e.g., a therapeutic composition or agent) that produces at least one desired therapeutic effect in a subject, such as preventing or treating a target condition or beneficially alleviating a symptom associated with the condition.

As used herein, a “VNAR domain” has the general structure, from N to C terminus, given by the formula FW1-CDR1-FW2-HV2-FW2′-HV4-FW3-CDR3-FW4, wherein the FWs are framework regions, CDRs are complementarity determining regions and HVs are hypervariable regions that collectively form the variable domain of a shark IgNAR (“VNAR”). The CDR1 and CDR3 from a VNAR that bind to, or are specific for, a particular antigen, when taken together are referred to herein as a cognate pair (or a CDR1/CDR3 cognate pair) for the given antigen. Because sharks produce different VNAR isoforms, CDR1/CDR3 cognate pairs are isoform specific. The three major VNAR isoforms are Type I, Type II and Type IV and are distinguishable by the number of disulfide bridges present in the domain, namely 3, 2 and 1 disulfide bridges, respectively. A fourth isoform, Type III isoform, is predominantly produced in neonatal animals.

As used herein, a “VNAR scaffold” refers to the structural framework regions (all FW and HV regions) of a VNAR domain other than CDR1 and CDR3 regions.

As used herein, the terms “VNAR antibody,” “VNAR-Fc fusion,” and “VNAR-Fc fusion protein” are used interchangeably, and include, but are not limited to antibodies that have a VNAR domain as their variable region and a non-IgNAR constant regions derived from the Fc fragments of IgG, IgM, IgA and IgE. In other words, the non-IgNAR constant region of a VNAR antibody includes the Fc portion of conventional antibodies, whether joined by chemical linkers or joined as fusion proteins with or without amino acid linking regions. Further, VNAR antibodies can be monovalent or bivalent. For the avoidance of doubt TXP1, A06 and G12 are VNAR antibodies which have VNAR domain and an Fc domain from human IgG.

As used herein, the term “TfR,” “TfR1” or “TfR-1” refers to a mammalian transferrin receptor-1 (in context as a protein or a nucleic acid), unless the context indicates that it refers specifically to human TfR-1 (see, e.g., UniProt P02786 TFR1_Human) or mouse TfR-1.

### Deimmunized VNAR Scaffolds and Domains

In accordance with the disclosure, one aspect described herein relates to deimmunized Type II VNAR domains and scaffolds.

To identify potential MHC class II binding site in VNAR scaffolds, the scaffold of TXP1 (a Type II VNAR antibody specific for TfR-1) was analyzed in silico for the presence of human T-cell epitopes and specific amino acid changes were made and tested to provide non-immunogenic, stable scaffolds that retained antigen binding. This analysis identified and confirmed the presence of three regions in Type II VNAR scaffolds with MHC binding sites that can be engineered for human compatibility. These regions are designated herein as (i) region 1 which comprises or consists of amino acid residues ITKETGESL (SEQ ID NO: 1), (ii) region 2 which comprises or consists of amino acid residues YVETVNSGSK (SEQ ID NO: 2) and (iii) region 3 which comprises or consists of is amino acid residues YGGGTAVTVN (SEQ ID NO: 3). Hence, the VNAR scaffolds of the disclosure may have from 1-5 amino acid substitutions, insertions or deletions per region provided that such alterations maintain the overall primary and tertiary structure of the Type II VNAR domain and do not create a human T-cell epitope.

Accordingly, in an embodiment, the disclosure provides a naturally occurring or artificial Type II VNAR scaffolds, containing one or more of these regions, which has been deimmunized by independently mutating any one, two or three, or any combination thereof, of the regions by substitution, deletion, or insertion of from one to three amino acids per region. In an exemplary embodiment, (i) region 1 is amino acid residues 10 to 18 of SEQ ID NO. 10 (i.e., residues ITKETGESL), (ii) region 2 is amino acid residues 55 to 64 of SEQ ID NO. 10 (i.e., residues YVETVNSGSK); and (iii) region 3 is amino acid residues 97 to 106 of SEQ ID NO. 10 (i.e., residues YGGGTAVTVN), when CDR1 has 8 amino acid residues and CDR3 has 12 amino acid residues, and wherein SEQ ID NO: 10 is shown below:

¹ARVDQTPQTITKETGESLTINCVLR-CDR1-³⁴TYWYRK
KSGSTNEENISKGGRYVETVNSGSKSFSLKINDLTVEDS
GTYRCN-CDR3-⁹⁷YGGGTAVTVNA.

The superscript amino acid numbering is based on the amino acid sequence for the VNAR domain of TXP1 (see, SEQ ID NO: 7), which has a CDR1 of 8 amino acids and a CDR3 of 12 amino acids. While CDR1 regions are typically 8 amino acids in a Type II VNAR, the CDR3 region can vary from about 7 to about 16 amino acids in length.

The VNAR scaffolds of the disclosure can be engineered into, for example, nucleic acid or polypeptide libraries and used to select for VNAR domains against target antigens of interest or to mature the antigen binding sites. The VNAR scaffolds can also be used to graft CDR1/CDR3 cognate pairs specific for a target antigen of interest, and conversely, a VNAR domain specific for a target antigen of interest can be deimmunized in accordance with the teachings herein. Hence, the VNAR scaffolds may be in a VNAR domain having a randomized CDR1 and CDR3, a known CDR1 and a randomized CDR3, a randomized CDR1 and a known CDR3, or a known CDR1 and CDR3. When the CDR1/CDR3 pair is known, i.e., it is a cognate pair for a target antigen of interest, it can be grafted into a deimmunized VNAR scaffold, or it can be part of a VNAR domain wherein the VNAR scaffold is being deimmunized. In other uses, such cognate pairs can be partially randomized or specifically mutated for paratope maturation to select for VNAR domains with higher affinity or other characteristics of interest.

Accordingly, some embodiments of the disclosure are directed to a Type II VNAR scaffold for a VNAR domain having a formula of, from N to C terminus,
FW1-CDR1-FW2-HV2-FW2′-HV4-FW3-CDR3-FW4
wherein the scaffold comprises an amino acid sequence in which

FW1 is
(SEQ ID NO: 4)
ARVDQTPQTITKETGESLTINCVLR;
HV2--FW2'--HV4--FW3 is
(SEQ ID NO: 5)
TYWYRKKSGSTNEENISKGGRYVETVNSGSKSFSLKINDL
TVEDSGTYRCN;
and
FW4 is
(SEQ ID NO. 6)
YGGGTAVTVNA,

wherein CDR1 and CDR3 taken together each have at least one cysteine residue; and
wherein at least one of regions 1, 2 or 3 has one, two or three amino acids changes to reduce or remove the immunogenicity of an MHC class II binding site,
wherein
   (i) region 1 is amino acid residues 10 to 18 of SEQ ID NO. 7 (i.e., residues ITKETGESL),
   (ii) region 2 is amino acid residues 55 to 64 of SEQ ID NO. 7 (i.e., residues YVETVNSGSK); and
   (iii) region 3 is amino acid residues 97 to 106 of SEQ ID NO. 7 (i.e., residues YGGGTAVTVN).

In some embodiments, the VNAR scaffold forms part of a VNAR domain comprising a CDR1 region having an amino acid sequence of DSNCALSS. In some embodiments, the VNAR scaffold forms part of a VNAR domain comprising a CDR3 region having an amino acid comprising from 7 to 25 amino acids, or having 7, 8, 9, 10, 11, 12, 13, 14, 15, 16, 17, 18, 19, 20, 21, 22, 23, 24 or 25 amino acids. In some embodiments, the CDR3 comprises from 8 to 14 amino acids. In some embodiments, the CDR3 comprises 12 amino acids.

Any of the foregoing VNAR scaffolds can form part of a VNAR domain of known specificity (i.e., when used with CDR1/CDR3 cognate pairs) or of unknown specificity (i.e., when used in a VNAR screening library).

Accordingly, in some embodiments, the disclosure provides a Type II VNAR domain comprising, from N to C terminus,

(SEQ ID NO: 10)
¹ARVDQTPQTITKETGESLTINCVLR-CDR1-³⁴TYWYRKKS
GSTNEENISKGGRYVETVNSGSKSFSLKINDLTVEDSGTYR
CN-CDR3-⁹⁷YGGGTAVTVNA,

wherein CDR1 and CDR3 are a Type II cognate pair for a target antigen of interest; and
wherein at least one of regions 1, 2 or 3 has one, two or three amino acids changes to reduce or remove the immunogenicity of an MHC class II binding site, wherein
   (i) region 1 is amino acid residues 10 to 18 of SEQ ID NO. 7 (i.e., residues ITKETGESL),
   (ii) region 2 is amino acid residues 55 to 64 of SEQ ID NO. 7 (i.e., residues YVETVNSGSK); and
   (iii) region 3 is amino acid residues 97 to 106 of SEQ ID NO. 7 (i.e., residues YGGGTAVTVN).

In accordance with the disclosure, the amino acid changes provided herein can be used with any of the VNAR scaffolds or VNAR domains of the disclosure.

In some embodiments, the amino acid change in region 1 is 110A, I10E, 110S, or I10T. As is well known in the art, using one-letter amino acid codes, these changes indicated that the first amino acid present at the indicated position has been changed to the second amino acid. For example, the designation 110A means that the isoleucine at position 10 has been changed to an alanine.

In some embodiments, the amino acid change in region 2 is (a) a one amino acid change of Y55H, Y55N, Y55S or Y55T, or (b) two amino acid changes of N60D and S63T, R54K and N60E, or R54K and N60Q.

In some embodiments, the amino acid change in region 3 is (a) a one amino acid change of Y97S, G99D or A102V, or (b) three amino acid changes of Y97S, G99D and A102V.

In some embodiments, any of the foregoing the amino acid can be present alone or in any combination with others from the same region or from different regions.

In some embodiments, at least one amino acid is changed in each of regions 1, 2 and 3. In exemplary embodiments, wherein at least one amino acid change is made in each of the three regions, such changes can be selected from the group consisting of
I10A, N60D, S63T, Y97S, G99D and A102V;
I10A, R54K, N60E, Y97S, G99D and A102V;
I10A, R54K, N60Q, Y97S, G99D and A102V;
I10A, Y55H, Y97S, G99D and A102V;
I10A, Y55N, Y97S, G99D and A102V;
I10A, Y55S, Y97S, G99D and A102V;
I10A, Y55T, Y97S, G99D and A102V;
I10S, Y55H, Y97S, G99D and A102V; and
I10T, Y55H, Y97S, G99D and A102V.

In some embodiments, the amino acid sequence between CDR1 and CDR3 is

(SEQ ID NO: 13)
TYWYRKKSGSTNEENISKGGRYVETVDSGTKSFSLKINDLT
VEDSGTYRCN
or
(SEQ ID NO: 14)
TYWYRKKSGSTNEENISKGGRKYVETVESGSKSFSLKINDL
TVEDSGTYRCN.

In any of the foregoing embodiments, the VNAR domain has CDR1/CDR3 cognate pairs that are specific for a target of interest. Such targets include, but are not limited to, a B-lymphocyte stimulator (BAFF), a transferrin receptor (TfR), CD98, any other another BBB receptor, or a COV2 spike protein. Additional targets of interest are provided below.

In some embodiments, a VNAR scaffold or a VNAR domain comprises an amino acid sequence shown in Table 1. These VNAR scaffolds are also referred to herein as r2d1-scaffold and r2d4 scaffold and the corresponding VNAR domains are referred to as VNAR-r2d1 and VNAR-r2d4, respectively. When these VNAR domains are fused to an Fc domain (in the same manner as described in Example 2), the resultant VNAR antibodies are referred to as R2D1 and R2D4, respectively. A similar nomenclature scheme is used throughout for other VNAR scaffolds, VNAR domains and VNAR antibodies of the disclosure. These VNARs scaffolds and domains are variants of VNAR-txp1 with deimmunized scaffolds, identified and obtained and characterized as described in U.S. Ser. No. 63/234,210, filed Aug. 17, 2021.

TABLE 1
Exemplary Deimmunized VNAR Scaffolds & Domains

| Name | Amino Acid Sequence |
|---|---|
| r2d1- | ARVDQTPQTATKETGESLTINCVLR-CDR1- |
| scaffold | TYWYRKKSGSTNEENISK |
| | GGRYVETVDSGTKSFSLKINDLTVEDSGTYRCN-CDR3- |
| | SGDGTVVTVNA (SEQ ID. NO: 15) |
| VNAR- | ARVDQTPQTATKETGESLTINCVLRDSNCALSSTYWYRKKSGSTNE |
| r2d1 | ENISKGGRYVETVDSGTKSFSLKINDLTVEDSGTYRCNVVGTWCMS |
| | WRDVSGDGTVVTVNA (SEQ ID. NO: 16) |
| r2d4- | ARVDQTPQTATKETGESLTINCVLR-CDR1- |
| scaffold | TYWYRKKSGSTNEENISK |
| | GGRKYVETVESGSKSFSLKINDLTVEDSGTYRCN-CDR3- |
| | SGDGTVVTVNA (SEQ ID. NO: 17) |
| VNAR- | ARVDQTPQTATKETGESLTINCVLRDSNCALSSTYWYRKKSGSTNE |
| r2d4 | ENISKGGRKYVETVESGSKSFSLKINDLTVEDSGTYRCNVVGTWCM |
| | SWRDVSGDGTVVTVNA (SEQ ID. NO: 18) |

The VNAR domains formed from the VNAR scaffolds of this disclosure can optionally have a His-Tag (or other convenient tag for purification purposes). In some cases, such tags are removable.

Examples of Type II CDR1s and Type II CDR3s are well know in the art and can be derived from any known Type II VNAR domain. Thus, the CDR1/CDR3 cognate pairs can be from known Type II VNAR domains, including for example those described in U.S. Pat. No. 10,435,474 (BAFF binders), U.S. Pat. No. 10,722,576 (TfR binders), Intl. Pub. No. WO2019/246288 (CD98 binders) or in U.S. Ser. No. 17/343,444 filed Jun. 9, 2021 (COV2 spike protein binders). In some embodiments the CDR1/CDR3 cognate pairs are from BBB-shuttling VNAR domains and include, but are not limited to, those pairs from (a) the TfR-binding VNAR domains designated as Clone C or one of its variants described in WO2018/031424 and WO2019/089395, respectively; (b) the TfR-binding VNAR domains designated as Clone H or one of its variants described in WO2018/031424 and WO2019/089395, respectively; (c) the TfR-binding VNAR domains designated as Clone 8 or one of its variants described in WO2020/056327; (d) TXB4 (also known as Clone 18 and described in WO2020/056327; (e) the CD98-binding VNAR domains described in WO2020/246288; (f) the TfR-binding VNAR domains described in WO2016/077840 as capable of BBB shuttling; (g) the TfR-binding VNAR domain variants of Clone C specifically designated as variants 7, 13, 14, 16, 18, 25, 30, 31, and 34 in WO2019/089395; and (h) the VNAR txp1 domain described herein and in U.S. provisional application U.S. Ser. No. 63/112,314, filed Nov. 11, 2020.

In accordance with the disclosure, the VNAR domains derived from the VNAR scaffolds include conjugates, nucleic acids encoding those VNAR domains and conjugates (as applicable) and methods of making the VNAR domains and conjugates as described hereinabove for the VNAR-txp1 domain.

Further, any VNAR domain formed from a VNAR scaffold of the disclosure can be used to form the variable domain of a single variable domain antibody, a bi- or tri-functional VNAR, a conventional antibody, or any fragment or fusion protein of said antibody as well as variable domains with antibody-like backbones. Such constructs can be made by methods known to those of skill in the art.

Examples of single variable domain antibodies include, but are not limited to, a shark or other cartilaginous fish antibodies, camelid antibodies and nanobodies. Examples of conventional antibodies (and their fragments) include, but are not limited to, immunoglobins having both heavy and light chains, such as IgM's, IgA's, IgG's, IgE's, single chain Fv's, Fab fragments, or any fragment or fusion protein of such antibodies or fragments.

In some embodiments, any of the VNAR domains formed from a VNAR scaffold of the disclosure can be fused to an Fc domain of a conventional antibody to form a VNAR-Fc conjugate, as described herein and includes but is not limited to, a human Fc domain (hFc), a cynomolgus macaque Fc domain (cFc) or a murine Fc domain. Most preferably the Fc domain is an hFc domain. In some embodiments, the Fc domain is from an IgG, and preferably from IgG1. In some embodiments, the VNAR domain is fused at the N-terminal end of an hFc IgG1, has attenuated effector function (AEF) and carries a series of mutations (E233P/L234V/L235A/AG236+A327G/A330S/P331S) (Lo 2017).

Non-limiting examples of antibody-like backbones that may be used according to the disclosure include monospecific and bispecific such as multimerizing scFv fragments (diabodies, triabodies, tetrabodies), disulfide stabilized antibody variable (Fv) fragments, disulfide stabilized antigen-binding (Fab) fragments consisting of the V_{L}, V_{H}, C_{L} and C_{H} 1 domains, bivalent F(ab′)2 fragments, Fd fragments consisting of the heavy chain and C_{H}1 domains, dimeric C_{H}2 domain fragments (C_{H}2D), Fc antigen binding domains (Fcabs), single chain Fv-C_{H}3 minibodies, bispecific minibodies, isolated complementary determining region 3 (CDR3) fragments, constrained FR3-CDR3-FR4 polypeptides, SMIP domains, and any genetically manipulated counterparts of the foregoing that retain TfR-1 binding function (see e.g., Weiner L, Cell 148:1081-4 (2012); Ahmad Z et al., Clin Dev Immunol 2012:980250 (2012) for reviews).

### Other Conjugates

In certain embodiments, a VNAR domain from a VNAR scaffold of the disclosure comprises a conjugate with a heterologous molecule or agent. Such conjugates can be used, for example, to target delivery of heterologous molecules across a membrane by association (e.g., a complex or conjugate), especially a BBB or a GI tract membrane. Such conjugates can also be used for diagnostic or detection purposes. with a VNAR domain of the disclosure. As described in more detail below, heterologous molecules may be selected from an enormously wide variety of agents.

Associated heterologous molecules which may be used in accordance with the disclosure may comprise, e.g., one or more biologically active molecules and/or imaging agents. Exemplary biologically active molecules include, e.g., toxins for targeted cell death (useful e.g., in certain hyperproliferative diseases or disorders such as cancers or aberrant proliferative conditions). Other exemplary biologically active molecules which may be transported in association with a VNAR domain of the disclosure include, e.g., polypeptides, such as an antibody or antibody fragment; a therapeutic peptide such as a hormone, cytokine, growth factor, enzyme, antigen or antigenic peptide, transcription factor, or any functional domain thereof. Other exemplary biologically active molecules which may be transported in association with a VNAR domain of the disclosure include, e.g., nucleic acid molecules, such as an oligonucleotide (e.g., single, double or more stranded RNA and/or DNA molecules, and analogs and derivatives thereof); small regulatory RNA such as shRNA, miRNA, siRNA and the like; and a plasmid or fragment thereof.

Exemplary polypeptides which may be therapeutically beneficial when administered as a heterologous molecule capable of crossing the BBB or other cell membrane include but are not limited to: a brain derived neurotrophic factor (BDNF), a bone morphogenic protein (e.g., BMP-1 through BMP-7, BMP8a, BMP8b, BMP10 and BMP15), a ciliary neurotrophic factor (CNF), an epidermal growth factor (EGF), erythropoietin, a fibroblast growth factor (FGF), a glial derived neurotrophic factor (GDNF), a heptocyte growth factor, an interleukin (e.g., IL-1, IL-4, IL-6, IL-10, IL-12, IL-13, IL-15, IL-17), a nerve growth factor (NGF), a neurotrophin (e.g., NT-3 and NT-4/5), a neurturin, a neuregulin, a platelet derived growth factor (PDGF), a transforming growth factor (e.g., TGF-alpha and TGF-beta), apolipoprotein E (ApoE), a vasoactive intestinal peptide, artemin, persephin, netrin, neurotensin, GM-GSF, cardiotrophin-1, stem cell factor, midkine, pleiotrophin, a saposin, a semaphorin, leukemia inhibitory factor, and the like.

Exemplary therapeutic antibodies or fragments that may be transported across the BBB or other cell membranes as a heterologous biologically active molecule include but are not limited to: antibodies for neurodegeneration including anti-Abeta, anti-Tau, anti-alpha-synuclein anti-Trem2, anti-C9orf7 dipeptides, anti-TDP-43, anti-prion protein C, anti-huntingtin, anti-nogo A, anti-TRAIL (tumor necrosis factor-related apoptosis-inducing ligand); antibodies for neuro-oncology including anti-HER2, anti-EGF, anti-PDGF, anti-PD1/PDL1, anti-CTLA-4, anti-IDO, anti-LAG-3, anti-CD20, anti-CD19, anti-CD40, anti-OX40, anti-TIM3, anti-toll-like receptors; antibodies for neuroinflammation including anti-TNF, anti-CD138, anti-IL-21, anti-IL-22; antibodies to viral diseases of the brain including anti-West Nile virus, anti-Zika, anti-HIV, anti-CMVanti-HSV and the like.

Exemplary enzymes that may be transported across the BBB or other cell membranes as a heterologous biologically active molecule include but are not limited to: alpha-L-iduronidase, iduronate-2-sulfatase, N-acetyl-galactosamine-6-sulfatase, arylsulfatase B, acid alpha-glucosidase, tripeptidyl-peptidase 1, acid sphingomyelinase glucocerebrosidase and heparan sulfamidase.

Also included as exemplary biologically active molecules are small molecules comprising chemical moieties (such as a therapeutic small molecule drugs); carbohydrates; polysaccharides; lipids; glycolipids and the like. Exemplary embodiments of such small molecule therapeutic agents include certain cancer drugs, such as daunorubicin, doxorubicin, and other cytotoxic chemical agents including microtubule inhibitors, topoisomerase inhibitors, platins, alkylating agents, and anti-metabolites all of which may beneficially be administered across the BBB at lower overall systemic doses than by IV administration. Other small molecule therapeutic agents may include corticosteroids, NSAIDs, COX-2 inhibitors, small molecule immunomodulators, non-steroidal immunosuppressants, 5-amino salicylic acid, DMARDs, hydroxychloroquine sulfate, and penicillamine. 1-D-ribofuranosyl-1,2,4-triazole-3 carboxamide, 9-2-hydroxy-ethoxy methylguanine, adamantanamine, 5-iodo-2′-deoxyuridine, trifluorothymidine, interferon, adenine arabinoside, protease inhibitors, thymidine kinase inhibitors, sugar or glycoprotein synthesis inhibitors, structural protein synthesis inhibitors, attachment and adsorption inhibitors, and nucleoside analogues such as acyclovir, penciclovir, valacyclovir, and ganciclovir, among others. Small molecule therapeutic agents which may be used according to the disclosure also include bevacizumab, cisplatin, irinotecan, methotrexate, temozolomide, taxol and zoledronate. Certain anti-inflammatory agents may be useful biologically active molecules. Fluoxetine, for example, reportedly inhibits MMP-2, MMP-9 and MMP-12 expression associated with blood-brain barrier disruption and inflammatory reactions after spinal cord injury, which may be used according to the disclosure to protect blood-brain barrier and to inhibit deleterious inflammatory responses in spinal cord injury and central nervous system disease. Other non-limiting examples of therapeutic antibodies which may be beneficially transported across the BBB include anti-CD133, anti-CD137, anti-CD27, anti-VEGF, anti-EGRFvIII, anti-IL-15 and anti-IL13R.

Exemplary embodiments of an imaging agent as an associated heterologous molecule include agents that comprise at least one of a metal such as a paramagnetic metal, a radionuclide such as a radioisotope, a fluorochrome or fluorophor, an energy emitting particle, a detectable dye, and an enzyme substrate.

Further examples of biologically active molecules include small molecules, including therapeutic agents, in particular those with low blood-brain barrier permeability. Some examples of these therapeutic agents include cancer drugs, such as daunorubicin, doxorubicin, and toxic chemicals which, because of the lower dosage that can be administered by this method, can now be more safely administered. For example, a therapeutic agent can include bevacizumab, irinotecan, zoledronate, temozolomide, taxol, methotrexate, and cisplatin.

In another embodiment, a therapeutic agent can include a broad-spectrum antibiotic (e.g., cefotaxime, ceftriaxone, ampicillin and vancomycin); an antiviral agent (e.g., acyclovir); acetazolamide; carbamazepine; clonazepam; clorazepate dipotassium; diazepam; divalproex sodium; ethosuximide; felbamate; fosphenytoin sodium; gabapentin; lamotrigine; levetiracetam; lorazepam; oxcarbazepine; phenobarbital; phenytoin; phenytoin sodium; pregabalin; primidone; tiagabine hydrochloride; topiramate; trimethadione; valproic acid; zonisamide; copaxone; tysabri; novantrone; donezepil HCL; rivastigmine; galantamine; memantine; levodopa; carbidopa; parlodel, permax, requip, mirapex; Symmetrel; artane; cogentin; eldepryl; and deprenyl. Antiviral compounds are also beneficial therapeutic agents that can be delivered using a VNAR domain of the disclosure, especially for cases in which the virus uses membrane transporter as its route of entry into infected cells, and that VNAR domain is specific for that membrane transporter.

Numerous other examples of biologically active molecules may be used in association with VNAR domain from a VNAR scaffold of the disclosure, appropriate selection of which will be apparent to the skilled artisan depending on the condition, disease or disorder to be treated.

Yet other examples of a biologically active molecule which may be used according to the present disclosure is an antigenic peptide. Antigenic peptides may provide immunological protection when imported by cells involved in an immune response. Other examples include immunosuppressive peptides (e.g., peptides that block autoreactive T cells, such peptides being known in the art).

An imaging agent, as used herein, may be any chemical substance which may be used to provide a signal or contrast in imaging. A signal enhancing domain may be an organic molecule, metal ion, salt or chelate, a particle (e.g., iron particle), or a labeled peptide, protein, glycoprotein, polymer or liposome. For example, an imaging agent may include one or more of a radionuclide, a paramagnetic metal, a fluorochrome, a dye, and an enzyme substrate.

For x-ray imaging, the imaging agent may comprise iodinated organic molecules or chelates of heavy metal ions of atomic numbers 57 to 83. In certain embodiments, the imaging agent is I¹²⁵ labeled IgG (see, e.g., M. Sovak, ed., “Radiocontrast Agents,” Springer-Verlag, pp. 23-125 (1984).

For ultrasound imaging, an imaging agent may comprise gas-filled bubbles or particles or metal chelates where the metal ions have atomic numbers 21-29, 42, 44 or 57-83. See e.g., Tyler et al., Ultrasonic Imaging, 3, pp. 323-29 (1981) and D. P. Swanson, “Enhancement Agents for Ultrasound: Fundamentals,” Pharmaceuticals in Medical Imaging, pp. 682-87. (1990) for other suitable compounds.

For nuclear radiopharmaceutical imaging or radiotherapy, an imaging agent may comprise a radioactive molecule. In certain embodiments, chelates of Tc, Re, Co, Cu, Au, Ag, Pb, Bi, In and Ga may be used. In certain embodiments, chelates of Tc-99m may be used. See e.g., Rayudu GVS, Radiotracers for Medical Applications, I, pp. 201 and D. P. Swanson et al., ed., Pharmaceuticals in Medical Imaging, pp. 279-644 (1990) for other suitable compounds.

For ultraviolet/visible/infrared light imaging, an imaging agent may comprise any organic or inorganic dye or any metal chelate.

For MRI, an imaging agent may comprise a metal-ligand complex of a paramagnetic form of a metal ion with atomic numbers 21-29, 42, 44, or 57-83. In certain embodiments, the paramagnetic metal is selected from: Cr(III), Cu(II), Dy(III), Er(III) and Eu(III), Fe(III), Gd(III), Ho(III), Mn(II and III), Tb(III). A variety of chelating ligands useful as MRI agents are well known in the art.

In some embodiments, the disclosure provides VNAR domains from the VNAR scaffolds of the disclosure comprising the VNAR domain operably linked to a heterologous molecule which differs in biological activity from the VNAR domain. Such operable linkages can be a covalent or non-covalent linkage and the heterologous molecule can be a growth factor, cytokine, lymphokine, cell surface antigen or an antibody or antibody fragment which binds to any of the foregoing; a chimeric antigen receptor; a cytotoxic small molecule; a biochemical pathway agonist or antagonist; a therapeutic agent or drug; a diagnostic agent such as a fluorescent molecule or other molecular marker; or a nucleic acid molecule with targeting or other regulatory properties (e.g., silencers) or which encodes a regulatory molecule for a cell.

In other aspects, the VNAR domains from the VNAR scaffolds of the disclosure may optionally be conjugated (e.g., using linkers such as chemical linkers and/or linker peptides which are not usually associated with the domains being associated) to one or more additional agents which may include therapeutic and/or diagnostic agents. Such agents include but are not limited to chemotherapeutics such as cytostatic drugs, cytotoxins, radioisotopes, chelators, enzymes, nucleases, nucleic acids such as DNA, RNA or mixed nucleic acid oligonucleotides, including siRNAs, shRNAs, microRNAs, aptamers and the like; immunomodulators such as therapeutic antibodies, antibody and antibody-like fragments, inflammatory and anti-inflammatory cytokines, anti-inflammatory agents, radiotherapeutics, photoactive agents, diagnostic markers and the like. In certain embodiments, the pharmaceutically active moieties of the disclosure comprise at least one scFv molecule that is operably linked via a linker peptide to the C-terminus and/or N-terminus of an Fc region.

In certain embodiments, the conjugates of the disclosure are multispecific, i.e., have at least one binding site that binds to a first molecule or epitope of a molecule and one or more other binding sites that bind to at least one heterologous molecule or to a different epitope or another molecule. Multispecific binding molecules of the disclosure may have specificity for at least two distinct binding sites, three distinct binding sites, four distinct binding sites or more. The disclosure further provides bivalent and multivalent conjugates comprising the VNAR domains from the VNAR scaffolds of the disclosure. Multivalent conjugates have two or more VNAR domains with the same specificity, with bivalent conjugates having two VNAR domains.

A large body of art is available relating to how to make and use antibody drug conjugates. See, e.g., WO2007/140371; WO2006/068867. Methods for peptide conjugation and for labeling polypeptides and conjugating molecules are well known in the art.

### Nucleic Acid Sequences That Encode a TfR Selective Binding Moiety

In one aspect, the disclosure provides an isolated nucleic acid which encodes a VNAR scaffold, VNAR domain, or conjugate of the disclosure, or a fragment or derivative thereof. The disclosure also provides an isolated nucleic acid molecule comprising a sequence that hybridizes under stringent conditions to those nucleic acids as well as the antisense or complement of any nucleic acids.

In another aspect, provided herein is an isolated nucleic acid molecule encoding a fusion protein comprising at least two segments, wherein one of the segments comprises a VNAR domain from the VNAR scaffolds of the disclosure. In certain embodiments, a second segment comprises a heterologous signal polypeptide, a heterologous binding moiety, an immunoglobulin fragment such as a Fc domain, or a detectable marker.

As used herein, the term “nucleic acid molecule” is intended to include DNA molecules, RNA molecules (e.g., mRNA, shRNA, siRNA, microRNA), analogs of the DNA or RNA generated using nucleotide analogs, and derivatives, fragments and homologs thereof. The nucleic acid molecules of the disclosure may be single-, double-, or triple-stranded. A nucleic acid molecule of the present disclosure may be isolated using sequence information provided herein and well known molecular biological techniques (e.g., as described in Sambrook et al., Eds., MOLECULAR CLONING: A LABORATORY MANUAL 2ND ED., Cold Spring Harbor Laboratory Press, Cold Spring Harbor, N.Y., 1989; and Ausubel, et al., Eds., CURRENT PROTOCOLS IN MOLECULAR BIOLOGY, John Wiley & Sons, New York, N.Y., 1993).

A nucleic acid molecule of the disclosure may be amplified using any form of nucleic acid template and appropriate oligonucleotide primers according to standard PCR amplification techniques. Amplified nucleic acid may be cloned into an appropriate vector and characterized, e.g., by restriction analysis or DNA sequencing. Furthermore, oligonucleotides corresponding to nucleotide sequences that encode a VNAR domain of the disclosure may be prepared by standard synthetic techniques, e.g., using an automated DNA synthesizer.

The term “oligonucleotide” as used herein refers to a series of covalently linked nucleotide (or nucleoside residues, including ribonucleoside or deoxyribonucleoside residues) wherein the oligonucleotide has a sufficient number of nucleotide bases to be used in a PCR reaction. Oligonucleotides comprise portions of a nucleic acid sequence having at least about 10 nucleotides and as many as 50 nucleotides, preferably about 15 nucleotides to 30 nucleotides. Oligonucleotides may be chemically synthesized and may be used as probes. A short oligonucleotide sequence may be used to amplify, confirm, or reveal the presence of an identical, similar or complementary DNA or RNA in a particular cell or tissue.

Derivatives or analogs of the nucleic acid molecules (or proteins) of the disclosure include, inter alia, nucleic acid (or polypeptide) molecules having regions that are substantially homologous to the nucleic acid molecules or proteins of the disclosure, e.g., by at least about 45%, 50%, 70%, 80%, 95%, 98%, or even 99% identity (with a preferred identity of 80-99%) over a nucleic acid or amino acid sequence of the same size or when compared to an aligned sequence in which the alignment is done by a computer homology program known in the art. A percent identity for any candidate nucleic acid or polypeptide relative to a reference nucleic acid or polypeptide may be determined by aligning a reference sequence to one or more test sequences using, for example, the computer program ClustalW (version 1.83, default parameters), which enable nucleic acid or polypeptide sequence alignments across their entire lengths (global alignment) or across a specified length. The number of identical matches in such a ClustalW alignment is divided by the length of the reference sequence and multiplied by 100.

Also included are nucleic acid molecules capable of hybridizing to the complement of a sequence encoding the proteins of the disclosure under stringent or moderately stringent conditions. See e.g. Ausubel, et al., CURRENT PROTOCOLS IN MOLECULAR BIOLOGY, John Wiley & Sons, New York, N.Y., 1993, and below. An exemplary program is the GAP program (Wisconsin Sequence Analysis Package, Version 8 for UNIX, Genetics Computer Group, University Research Park, Madison, Wis.) using the default settings, which uses the algorithm of Smith and Waterman (1981) Adv. Appl. Math. 2:482489). Derivatives and analogs may be full length or other than full length, if the derivative or analog contains a modified nucleic acid or amino acid, as described below.

Stringent conditions are known to those skilled in the art and may be found in CURRENT PROTOCOLS IN MOLECULAR BIOLOGY, John Wiley & Sons, N.Y. (1989), 6.3.1-6.3.6. In certain embodiments, stringent conditions typically permit sequences at least about 65%, 70%, 75%, 85%, 90%, 95%, 98%, or 99% homologous to each other to remain hybridized to each other. A non-limiting example of stringent hybridization conditions is hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C. This hybridization is followed by one or more washes in 0.2×SSC, 0.01% BSA at 50° C. The term “stringent hybridization conditions” as used herein refers to conditions under which a nucleic acid probe, primer or oligonucleotide will hybridize to its target sequence, but only negligibly or not at all to other nucleic acid sequences. Stringent conditions are sequence- and length-dependent and depend on % (percent)-identity (or %-mismatch) over a certain length of nucleotide residues. Longer sequences hybridize specifically at higher temperatures than shorter sequences. Generally, stringent conditions are selected to be about 5° C. lower than the thermal melting point (Tm) for the specific sequence at a defined ionic strength and pH. Stringent conditions may also be achieved with the addition of destabilizing agents, such as formamide.

### Methods of Producing TfR-Specific VNAR Domains and Conjugates

The VNAR domain from the VNAR scaffolds and the corresponding conjugates of the disclosure may be manufactured by standard synthetic methods, by use of recombinant expression systems, or by any other suitable method. Thus, these compounds may be synthesized in a number of ways, including, e.g., methods comprising: (1) synthesizing the polypeptide or polypeptide component of such VNAR domains using standard solid-phase or liquid-phase methodology, either stepwise or by fragment assembly, and isolating and purifying the final peptide compound product; (2) expressing a nucleic acid construct that encodes the polypeptide or polypeptide component of such VNAR domains in a host cell and recovering the expression product from the host cell or host cell culture; or (3) cell-free in vitro expression of a nucleic acid construct encoding the polypeptide or polypeptide component of such VNAR domains, and recovering the expression product; or by any combination of the methods of (1), (2) or (3) to obtain fragments of the peptide component, subsequently joining (e.g., ligating) the fragments to obtain the peptide component, and recovering the peptide component.

It may be preferable to synthesize a polypeptide or polypeptide component of such VNAR domains by means of solid-phase or liquid-phase peptide synthesis and such may be suitably accomplished by standard synthetic methods. Thus, peptides may be synthesized by, e.g., methods comprising synthesizing the peptide by standard solid-phase or liquid-phase methodology, either stepwise or by fragment assembly, and isolating and purifying the final peptide product. In this context, reference may be made to WO1998/11125 or, inter alia, Fields, G. B. et al., “Principles and Practice of Solid-Phase Peptide Synthesis”; in: Synthetic Peptides, Gregory A. Grant (ed.), Oxford University Press (2nd edition, 2002) and the synthesis examples herein.

Accordingly, the present disclosure also provides methods for producing a VNAR domain from the VNAR scaffolds of the disclosure according to above recited methods; a nucleic acid molecule encoding part or all of such polypeptides, a vector comprising at least one nucleic acid of the disclosure, expression vectors comprising at least one nucleic acid of the disclosure capable of producing a polypeptide of the disclosure when introduced into a host cell, and a host cell comprising a nucleic acid molecule, vector or expression vector of the disclosure.

Such VNAR domains may be prepared using recombinant techniques well known in the art. In general, methods for producing polypeptides by culturing host cells transformed or transfected with a vector comprising the encoding nucleic acid and recovering the polypeptide from cell culture are described in, e.g., Sambrook et al., Molecular Cloning: A Laboratory Manual (New York: Cold Spring Harbor Laboratory Press, 1989); Dieffenbach et al., PCR Primer: A Laboratory Manual (Cold Spring Harbor Laboratory Press, 1995).

A nucleic acid encoding a desired polypeptide may be inserted into a replication vector for further cloning (amplification) of the DNA or for expression of the nucleic acid into RNA and protein. A multitude of cloning and expression vectors are publicly available.

Expression vectors capable of directing transient or stable expression of genes to which they are operably linked are well known in the art. The vector components generally include, but are not limited to, one or more of the following: a heterologous signal sequence or peptide, an origin of replication, one or more marker genes, an enhancer element, a promoter, and a transcription termination sequence, each of which is well known in the art. Optional regulatory control sequences, integration sequences, and useful markers that can be employed are known in the art.

Any suitable host cell may be used to produce the VNAR domains from the VNAR scaffolds of the disclosure. Host cells may be cells stably or transiently transfected, transformed, transduced or infected with one or more expression vectors which drive expression of a polypeptide of the disclosure. Suitable host cells for cloning or expressing nucleic acids of the disclosure include prokaryote, yeast, or higher eukaryote cells. Eukaryotic microbes such as filamentous fungi yeast, Arabidopsis, and other plant and animal eukaryotic host cells that may be grown in liquid culture are suitable cloning or expression hosts for vectors. Suitable host cells for the expression of glycosylated polypeptides may also be derived from multicellular organisms.

Creation and isolation of host cell lines producing such VNAR domains or conjugates can be accomplished using standard techniques known in the art. Mammalian cells are preferred host cells for expression of peptides. Particularly useful mammalian cells include, inter alia, HEK 293, NSO, DG-44, and CHO cells, but any other suitable host cell may be used according to the disclosure. Those VNAR domains or conjugates are secreted into the medium in which the host cells are cultured, from which the VNAR domains or conjugates may be recovered or purified.

When a polypeptide is produced in a recombinant cell other than one of human origin, it is typically free of polypeptides of human origin. In certain embodiments, it is advantageous to separate a polypeptide away from other recombinant cell components such as host cell polypeptides to obtain preparations that are of high purity or substantially homogeneous. As a first step, culture medium or cell lysates may be centrifuged to remove particulate cell debris and suitable protein purification procedures may be performed. Such procedures include, inter alia, fractionation (e.g., size separation by gel filtration or charge separation by ion-exchange column); ethanol precipitation; Protein A Sepharose columns to remove contaminants such as IgG; hydrophobic interaction chromatography; reverse phase HPLC; chromatography on silica or on cation-exchange resins such as DEAE and the like; chromatofocusing; electrophoretic separations; ammonium sulfate precipitation; gel filtration using, for example, Sephadex beads such as G-75. Any number of biochemical purification techniques may be used to increase the purity of a VNAR domain from a VNAR scaffold of the disclosure or conjugates thereof.

### Methods of Deimmunizing VNAR Scaffolds

A further aspect of the disclosure relates to methods of deimmunizing VNAR scaffolds which contain MHC class II binding sites. In some embodiments, potential T-cell epitopes in natural and/or artificial VNAR scaffolds are identified by in silico analysis of their amino acid sequences for the presence of MHC class II (MHCII) binding sites. Such analysis can be done for example, as described in Examples 4 and 5. Once these binding sites are identified, key amino acids within the binding site are changed to abrogate to MHC class II (MHCII) binding. To confirm such changes are effective, VNAR domains with those changes are synthesized and functionally evaluated using a variety of techniques know to those of skill in the art to establish that the VNAR domains are non-immunogenic and have retained antigen binding capacity. approach provides the foundation for the deimmunization of shark VNAR scaffolds. Hence, these VNAR domains have VNAR scaffolds with from 1-5 amino acid substitutions, insertions or deletions per region of MHCII binding sites provided that such alterations maintain the overall primary and tertiary structure of the Type II VNAR domain and do not create a human T-cell epitope.

In an some embodiment, the disclosure provides a method deimmunizing a VNAR scaffold which comprises introducing one or more a deimmunizing mutations at an MHC Class II binding site in regions 1, 2 or 3 or any combination thereof in a naturally occurring or artificial VNAR scaffold comprising one or more of those regions, wherein (i) region 1 comprises or consists of amino acid residues ITKETGESL (SEQ ID NO: 1), (ii) region 2 comprises or consists of amino acid residues YVETVNSGSK (SEQ ID NO: 2) and (iii) region 3 comprises or consists of is amino acid residues YGGGTAVTVN (SEQ ID NO: 3). The one or more deimmunizing mutations may include the VNAR scaffolds of the disclosure may have from 1-5, and preferably from 1-3, amino acid substitutions, insertions or deletions per region provided that such alterations maintain the overall primary and tertiary structure of the Type II VNAR domain and do not create a human T-cell epitope.

In some embodiments, the mutated VNAR scaffold is a mutated VNAR-txp1. In some embodiments, the mutations are the changes to regions 1, or 3 described hereinabove in the Detailed Description section entitled “Deimmunized VNAR Scaffolds and Domains.”

While some embodiments of the disclosure have been described by way of illustration, it will be apparent that the disclosure can be put into practice with many modifications, variations and adaptations, and with the use of numerous equivalents or alternative solutions that are within the scope of persons skilled in the art, without departing from the spirit of the disclosure or exceeding the scope of the claims.

All publications, patents, and patent applications are herein incorporated by reference in their entirety to the same extent as if each individual publication, patent or patent application was specifically and individually indicated to be incorporated by reference in its entirety.

### EXAMPLES

The examples presented herein represent certain embodiments of the present disclosure. However, it is to be understood that these examples are for illustration purposes only and do not intend, nor should any be construed, to be wholly definitive as to conditions and scope of this disclosure. The examples were carried out using standard techniques, which are known and routine to those of skill in the art, except where otherwise described in detail.

### Example 1. Production and Purification of Human Fc Fusion VNAR Antibody, TXP1

Selected VNARs obtained as described in PCT/US2021/058661, filed Nov. 9, 2021 (WO2022/103769) were synthesized and cloned into an expression vector containing human IgG1 Fc domain. The final VNAR antibody constructs were bivalent molecules with the VNAR domain fused to the N-terminus of a hFc domain with effector mutations (Lo 2017).

The VNAR-hFc formats were produced with VNARs at N-terminal end of hFc IgG1. A hFc domain with attenuated effector function (AEF) that carries a series of mutations (E233P/L234V/L235A/AG236+A327G/A330S/P331S) (Lo 2017) was used for all constructs. The Exp293F expression system (Thermo Fisher) was used for protein production following the manufacturer's instructions. After 5 days growth, the cells were centrifuged at 2,000 rpm for 10 min. Supernatants were filtered using 0.22 μm membranes and loaded onto HiTrap MabSelect SuRe column (GE Healthcare) pre-equilibrated against PBS, pH 7.4. Protein A affinity bound proteins were eluted with 0.1 M glycine, pH 3.5 and the buffer exchanged to PBS, pH 7.4 using HiPrep 26/10 Desalting column (GE Healthcare). Protein purity was assessed by analytical size exclusion chromatography (SEC) and sodium dodecyl sulfate polyacrylamide gel electrophoresis (SDS-PAGE). Endotoxins level test was carried out using Endotoxin LAL assay with Endosafe nexgen-PTS kit at 5-0.05 EU/ml sensitivity range.

Purified VNAR-hFc antibodies were extensively characterized and the VNAR-hFc named TXP1 was further characterized. The amino acid sequence of the VNAR domain thereof (designated herein as VNAR-txp1) is

(SEQ ID NO: 7)
ARVDQTPQTITKETGESLTINCVLRDSNCALSSTYWYRKKSGS
TNEENISKGGRYVETVNSGSKSFSLKINDLTVEDSGTYRCNVV
GTWCMSWRDVYGGGTAVTVNA.

The amino acid sequences of the CDR1, HV2, HV4, and CDR3 regions of VNAR-txp1 and the variants described below are provided in Table 2.

TABLE 2
Amino Acid Sequences of Specific
VNAR-txp1 Regions

| Name | CDR1 | HV2 | HV4 | CDR3 |
|---|---|---|---|---|
| VNAR-txp1 | DSNCALSS | TNEENISKG | SGSKS | VVGTWCMSWRDV |
| & variants | (SEQ ID | (SEQ ID | (SEQ ID | (SEQ ID |
| | NO. 8) | NO. 9) | NO. 11) | NO. 12) |

### Example 2. TXP1 Cross Reactivity and Binding Kinetics

TfR-1 binding ELISA. Nunc MaxiSorp plates were coated with 100 μl of 1 μg/ml of in-house purified recombinant human, mouse, rat and cynomolgus TfR-1 and incubated at 4° C. overnight. Plates were incubated with blocking buffer (2.5% non-fat dry milk in PBST) for 1 h at RT. Serially-diluted TXP1 was mixed with non-fat dry milk in PBST to a final concentration of 2.5% and incubated for 30 min. Blocked TXP1 solutions (100 μl) were transferred to the blocked plates and incubated for 1 hr. Plates were washed with PBST and incubated with anti-hFc HRP-conjugated antibody diluted 1:5,000 (Sigma) in blocking buffer for 30 min. Plates were washed and developed with SureBlue™ TMB substrate, the reaction stopped with 1% HCl and absorbance measured at 450 nm.

Binding kinetics. Binding kinetics of TXP1 and A06 VNAR antibodies were determined by surface plasmon resonance (SPR) using a Biacore T200 (GE Healthcare). A His-capture kit (GE Healthcare) was used to immobilize anti-His antibodies on CM5 chips (as recommended by the manufacturer). His-tagged recombinant cynomolgus and human TfR-1 in 0.1% BSA in HBS-EP+ buffer (GE Healthcare) was captured at flow rate 10 μl/min. Analyte binding was measured using the single cycle kinetic SPR method on a Biacore T200. Analytes were injected at increasing concentrations (0.98, 3.9, 15.6, 62.5 and 250 nM) in HBS-EP+ at flow rate 30 μl/min. A flow cell without TfR-1 captured served as a reference. The chips were regenerated in 10 mM glycine-HCl, pH 1.5. Sensorgrams were fitted using 1:1 binding model and kinetic constants determined using Biacore T200 Evaluation software (GE Healthcare). Association and dissociation were measured for 360s and 1500s respectively, with flow rate at 30 ml/min.

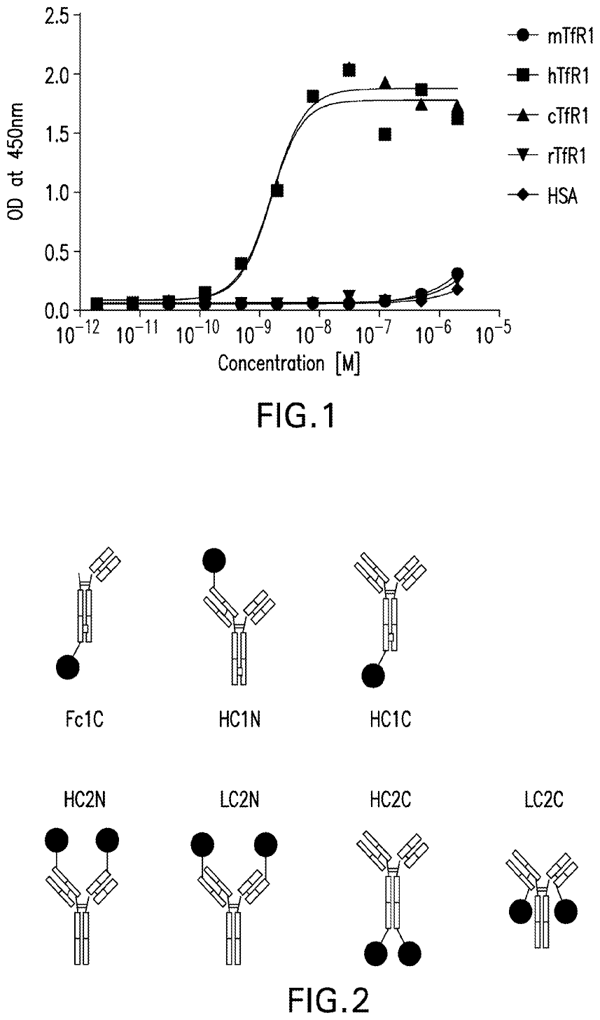

Results. Binding of TXP1 to recombinant human, cynomolgus, mouse and rat TfR-1 by ELISA showed cross-species reactivity to human and cynomolgus TfR-1 (FIG. 1) with EC50s of 1.5 nM and 1.6 nM for human and cynomolgus TfR-1, respectively (Table 3). No binding was observed for mouse or rat TfR-1 or for HSA, which was used as negative control.

TABLE 3
ELISA EC50 values of TXP1 for binding various species of TfR-1.

| EC50 [M] | mTfR-1 | hTfR-1 | cTfR-1 | rTfR-1 | HSA |
|---|---|---|---|---|---|
| TXP1 | NB | 1.5E−09 | 1.6E−09 | NB | NB |

NB: no binding

Binding kinetics assessed by surface plasmon resonance showed the affinity (KD) of TXP1 for human and cynomolgus TfR-1 to be approximately 1 nM and 3.1 nM, respectively (Table 4). The affinity obtained for the VNAR antibody A06 used as a comparator were approximately 3 nM and 2.6 nM for human and cynomolgus TfR-1, respectively. In Table 4, “ka” is the association rate constant, “kd” is the dissociation rate constant, and “KD” is the binding affinity.

TABLE 4
TXP1 and control A06 binding kinetics
to human and cynomolgus TfR-1.

| | | ka [1/Ms] | kd [1/s] | KD [M] |
|---|---|---|---|---|
| TXP1 | hTfR-1 | 1.29E+05 | 1.34E−04 | 1.03E−09 |
| | cTfR-1 | 1.16E+05 | 3.65E−04 | 3.14E−09 |
| A06 | hTfR-1 | 1.85E+06 | 5.47E−04 | 2.95E−10 |
| | cTfR-1 | 2.66E+05 | 7.01E−04 | 2.63E−09 |

### Example 3. TXP1-Mediated In Vivo Transport of a Therapeutic Antibody Across the Blood Brain Barrier

Brain shuttling efficacy of TXP1 can also be tested by genetically fusing its VNAR domain (VNATR-txp1) to different therapeutic antibodies. Rituximab (RIT), bapineuzumab (BAPI) and durvalumab (DUR) can be used as model antibodies in different mono- and bi-valent formats (FIG. 12). Injection into macaques into mice was performed as described in the below.

Two cynomolgus macaques per group are dosed with 1.35 mg/kg, IV. Blood and CSF are collected 20 hours post injection and brains were extracted following cardiac prefusion.

Balb/c mice (6-8 weeks) are injected intravenously with from 12.5 nmol/kg (0.9375 mg/kg) of protein to 25 nmol/kg and a blood sample is taken after 18 h. The animals are then perfused, and the brains are dissected and stored frozen. The whole brains are homogenized in 1% Triton X-100 and used for ELISA with anti-Fc capture and detection antibody. Standard curves can be prepared individually for each of the molecules to assure accuracy of the calculated concentrations. R3D11 and 1A can serve as negative VNAR-Fc controls that bind at nM concentration to TfR-1 but lack a blood brain penetration property.

### Example 4. Deimmunized VNAR Scaffolds

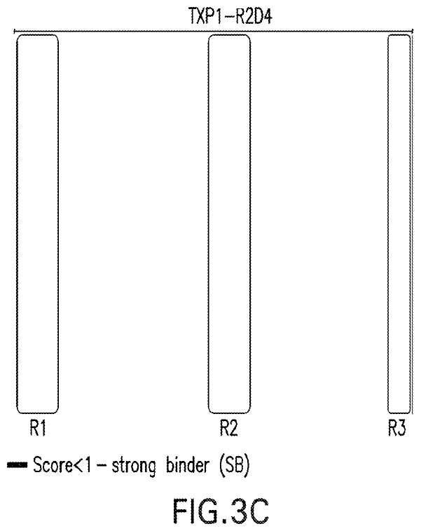

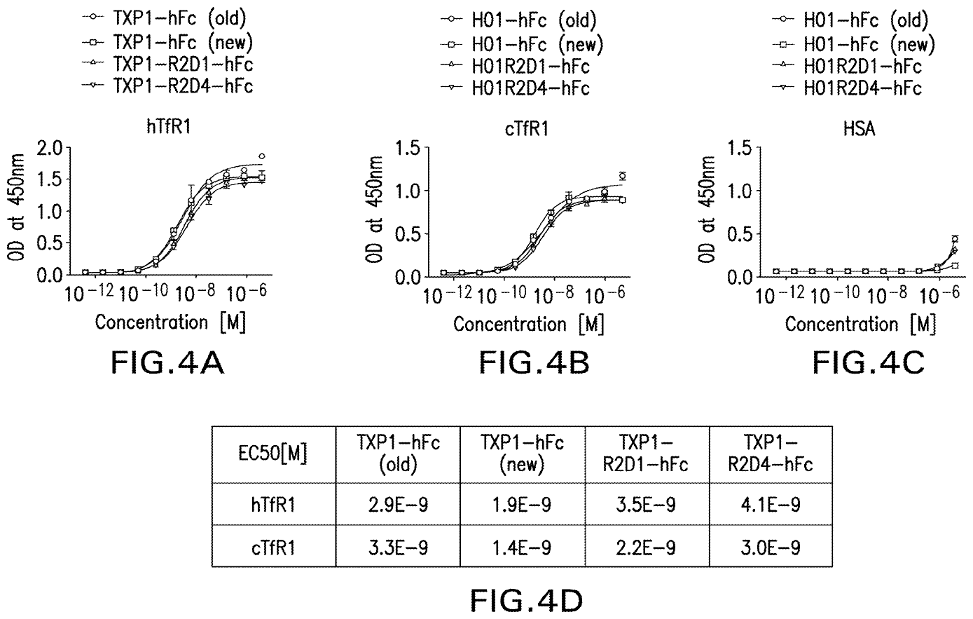

The VNAR scaffold from VNAR-txp1 was analysed in silico to identify human T-cell epitopes (Reynisson 2020) and possible amino acid substitutions for those positions in those epitopes were determined from a next generation sequence (NGS) databank from shark immune libraries. VNAR-txp1 variants with the selected non-immunogenic substitutions were prepared and analysed for binding activity and stability. The amino acid sequences of two exemplary variants are shown in Table 1. These deimmunized TXP1 variants R2D1 and R2D4 show no strong HQ HLA class II binders (FIG. 3) and no loss of TfR binding activity by ELISA (FIG. 4).

### Example 5. Removal of Potential Human T-Cell Epitope from TXP1

Results. The sequence of TXP1 was subjected to in silico analysis for possible motifs enabling MHCII class II presentation and subsequent T cell activation. The 53 most frequent MHCII alleles with known binding motifs were selected for analysis using the online tool NetMHCIIpan-4.0 [Reynisson 2020]. Starting from the first amino acid (aa), each subsequent peptide was offset by 1aa with a 14aa overlap, producing a total of 93 15-mer peptides for analysis.

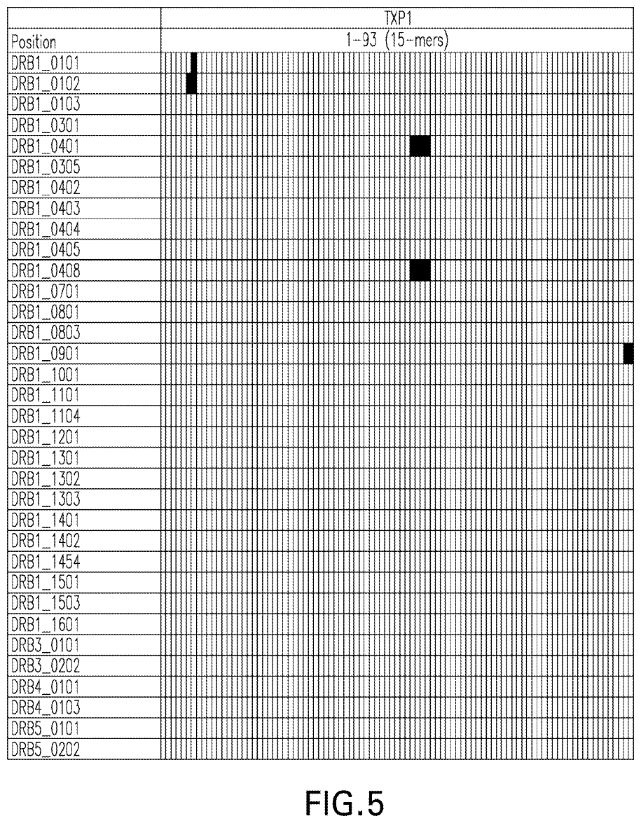

Sixteen MHCII class II alleles out of 53 tested were identified with the potential to present 15-mer peptides derived from TXP1 (FIG. 5). The binding strength score for these peptides was <1, which indicated a good match to the optimal motif for high affinity binding. Out of 40 peptides in total that were individually assigned to each of the tested MHCII allele, only 10 were unique due to similarities of binding motifs of some MHCII alleles. Three main regions (designated 1, 2 and 3) with potential immunogenic peptides were identified by mapping onto VNAR scaffold of the TXP1 sequence (FIG. 5). Region 1 encompasses residues from position 10 to 18 of SEQ ID NO: 7, region 2 encompasses residues from position 55 to 64 of SEQ ID NO: 7, and region 3 encompasses residue from position 97 to 106 of SEQ ID NO: 7.

Amino acid diversity at each position was determined from an internal database generated by next generation sequencing of naïve and immune libraries and contains over 150,000 unique type 2 VNAR frameworks. To minimize protein dysfunction, changes were considered based on the residue LOGO frequency [Crooks 2004], its position in the three-dimensional structure [Stanfield 2007] and its physio-chemical properties using the BLOSUM62 alignment matrix [Henikoff 1992]. All amino acid changes were then re-analyzed using NetMHCIIpan-4.0 to determine if a specific MHC class II binding motif was removed without introducing a new binding motif to a different allele.

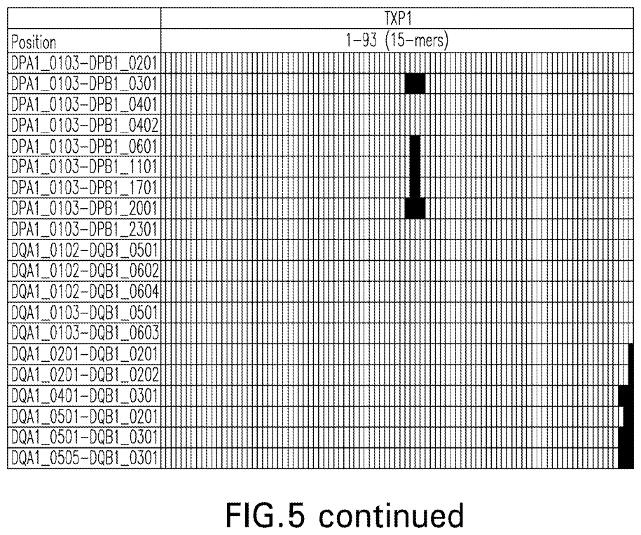

Mutating the primary anchoring residue in each MHCII binding motif was prioritized to minimize manipulation of the sequence with maximal reduction in binding score. Alternative positions were also considered when replacement of an anchor residue might disrupt the protein structure or where removing the anchor residue was insufficient to eliminate MHCII binding. Based on the in silico analyses, 20 single-region deimmunized variants of TXP1 (6 in region 1, 10 in region 2, and 4 in region 3) were genetically synthesized and evaluated (Table 5). Expression and hTfR1 binding were measured in crude media from transfected ExpiHEK cells (FIG. 6). Only the 4 single substitutions for region 1 and the 7 variants for region 2 were expressed and showed binding with >0.5-fold relative to parental TXP1. Region 3 required all three substitutions to eliminate predicted MHCII binding, and all three mutations individually and when combined (TXP1-R3-1-4) were expressed well and retained parental levels of TfR-1 binding.

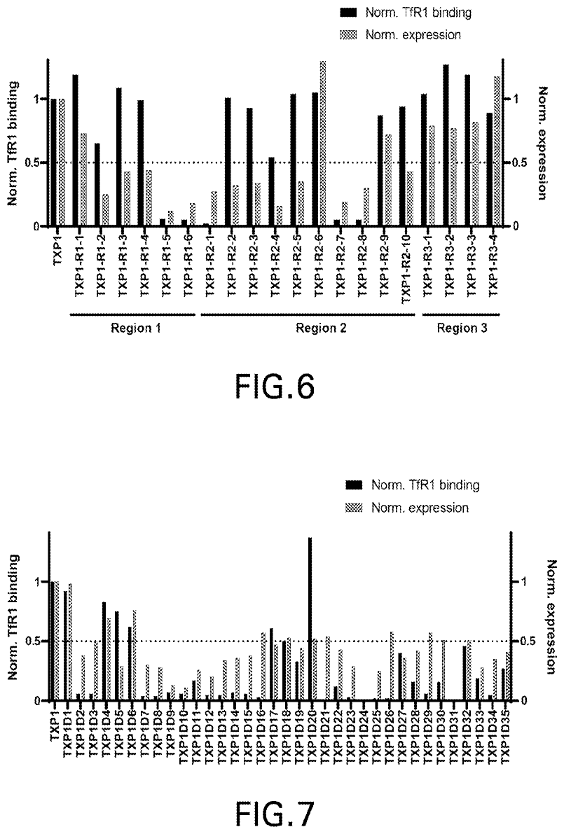

Single-region mutations were combined to create variants with all three regions deimmunized (Table 5). A total of 35 of fully-deimmunized variants were genetically synthesized and hTfR1 binding and expression were measured in crude media from transfected ExpiHEK cells (FIG. 7). Only 10 of the 35 combined variants retained binding to TfR1 with a 0.5-fold difference relative to TXP1. Attrition was mainly due to the impact of an inactive mutation used in multiple combinations. Of the 10 active variants, 9 contained the 6 mutations found to disrupt all 3 regions (a single mutation in region 1, a double mutation in region 2 plus a triple mutation in region 3), however, one variant (TXP1D6) carried 8 mutations (a triple instead of a single mutation in region 1) and was still expressed and bound TfR1.

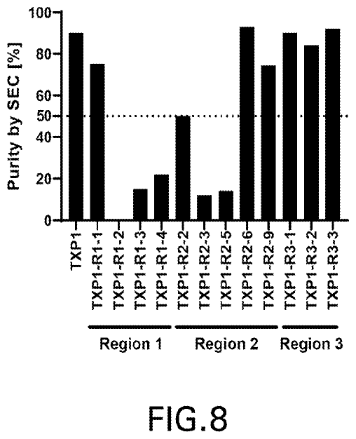

The twelve functional single-region variants of TXP1 were expressed at the 30-ml scale and purified by Protein A chromatography for further characterization. By analytical SEC (FIG. 8), some expressed variants showed significant protein misfolding or aggregation. A single variant in region 1 (TXP1-R1-1), 2 variants in region 2 (TXP1-R2-6 and TXP1-R2-9) and the 3 variants in region 3 showed the level of monomer purity of the parental TXP1. Substitutions at P55 in region 2, either alone (TXP1-R2-1-5) or in combination (TXP1D17-20), were poorly expressed (FIGS. 6 and 7) or produced low amounts of pure monomer despite good TfR1 binding (FIG. 8). This was likely a consequence of protein misfolding due to the location of P55 within the beta sheet structure. Similarly, TXP1D5 (N60Q substitution) and TXP1D6 (triple region 1 mutation) were expressed and bound TfR1 but showed significant aggregation when further analyzed by non-reducing SDS-PAGE followed by Western blotting (data not shown).

Two fully deimmunized variants, TXPID1 and TXP1D4 that differed only in modifications in region 2, had very similar properties to parental TXP1 and were produced at the 100-ml scale for further evaluation. While expression and purification parameters for TXPID1 and TXP1D4 were like parental TXP1, both variants showed higher expression yields and lower purity as determined by analytical SEC (Table 6). However, after a two-step purification with the addition of preparative SEC, the final purification yields of TXP1 and TXP1D4 were similar, whereas the yield of TXP1-D4 was approximately 60% higher than the parental TXP1. Melting temperatures (approximately 61° C.) were similar for TXP1, TXP1D1 and TXP1D4 (Table 6), indicating that the introduced mutations did not affect antibody stability under heat stress.

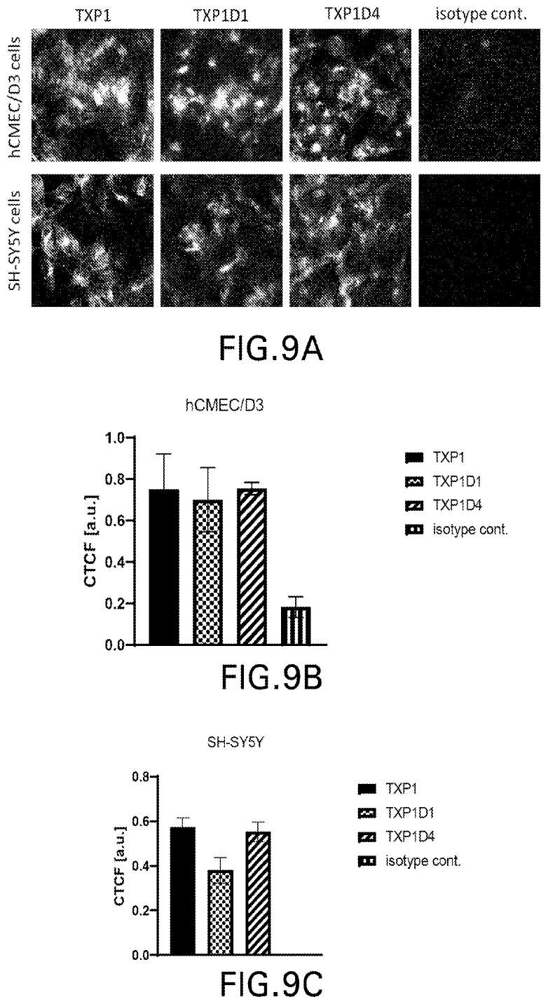

Binding of TXP1D1 and TXP1D4 to hTfR1 and cTfR1 by ELISA showed very similar low nM EC50 values to TXP1 (Table 6). The binding kinetics (affinity, on- and off-rates) as measured by SPR were also very similar between TXP1D1, TXPID4 and TXP1, with affinities of approximately 3-5 nM for hTfR1 and 2 nM cTfR1. Functional activity of the fully deimmunized variants was assessed using the hCMEC/D3 human endothelial cell line and the SY-SH5Y human neuronal cell line. Binding to hCMEC/D3 cells was assessed by incubation with serial antibody dilutions for 1h on ice to avoid internalization. The cells were fixed and binding to cell surface TfR1 was measured by flow cytometry. Cell binding EC50 values for TXP1, TXPID1 and TXP1D4 of approximately 2.2 nM, 1.6 nM and 2.1 nM, respectively were very similar (Table 6). Internalization via cell-surface hTfR1 was assessed using the hCMEC/D3 and SY-SH5Y cell lines by incubation with the antibodies for 1 hour at 37° C. Fluorescent microscopy showed a similar level of internalization between TXP1 and the deimmunized variants in both cell lines (FIG. 9).

### Methods

Production of VNAR antibodies. VNAR-hFc formats were produced with VNARs at N-terminal end of hFc IgG1 as described before [Stocki 2021]. The Exp293F expression system (Thermo) was used for protein production following the manufacturer's manual. After 5 days growth, the cells were centrifuged at 2,000 rpm for 10 min. Supernatants were filtered using 0.22 μm membranes and loaded onto HiTrap MabSelect SuRe column (Cytiva) pre-equilibrated against PBS, pH 7.4. Protein A affinity bound proteins were eluted with 0.1 M Glycine, pH 3.5 and the buffer exchanged to PBS, pH 7.4 using HiPrep 26/10 Desalting column (Cytiva). Protein purity was assessed by analytical size exclusion chromatography (SEC) and sodium dodecyl sulfate polyacrylamide gel electrophoresis (SDS-PAGE).

TfR1 ELISA. Nunc MaxiSorp plates were coated with 100 ul of 1 μg/ml of in-house purified recombinant human, cynomolgus TfR1 or HSA and incubated at 4° C. overnight. Plates were incubated with blocking buffer (2.5% non-fat dry milk in PBST) for 1 hr at RT. Serial dilutions of TXP1, TXPID1 or TXP1D4 were mixed with non-fat dry milk in PBST to a final concentration of 2.5% and incubated for 30 min. Blocked VNAR antibody solutions (100 μl) were transferred to the blocked plates and incubated for 1 hr. Plates were washed with PBST and incubated with anti-hFc HRP-conjugated antibody diluted 1:5,000 (Sigma) in blocking buffer for 30 min. Plates were washed and developed with SureBlue TMB substrate. Were indicated the velocity of colorimetric reaction development was measured in the kinetic mode at 370 nm and the maximum peak was used for quantitation (Vmax at 370 nm). Alternatively, the TMB reaction was stopped with 1% HCl and absorbance was measured at 450 nm (OD at 450 nm).

hFc ELISA. Nunc MaxiSorp plates were coated with 100 μl of mouse anti-Human IgG-CH2 domain (Thermo #MA5-16929) diluted 1:1000 in PBS overnight at 4° C. Plates were washed and incubated with blocking buffer for 1 hr at RT. Expression media was diluted 500-2000× in PBS before adding 100 μl to the ELISA plates and incubated for 1 hr at RT. Plates were washed with PBST and incubated with a goat anti-human Fc-peroxidase antibody diluted 1:5000 (Sigma) in blocking buffer for 1 hr. Plates were washed and developed with SureBlue™ TMB substrate, the reaction stopped with 1% HCl and absorbance measured at 450 nm. The VNAR-Fc concentration was determined from standard curves.

Analytical SEC. Protein samples were diluted in PBS pH 7.4 to the final concentration of 0.5 mg/ml and 50 μl samples were loaded onto PBS pH 7.4 pre-equilibrated column Superdex 200 Increase 10/300 (Cytiva) connected to AKTA PURE at room temperature with a flow rate of 0.75 ml/min. The UV signal was measured over the elution volume. MW markers were run before and after the samples monitor for any inconsistencies and mass shifts.

Thermal shift assay. Melting temperatures (Tm) of TXP1, TXP1D1 and TXP1D4 were assessed using thermal shift assay. The antibodies were diluted to 1 mg/ml in PBS and subsequently mixed with Sypro Orange (Thermo #S6650). The samples were loaded onto the plate and analyzed using Bio-Rad CFX instrument. The temperature gradient 20-95° C. was set to increase at 1° C. per minute with real time fluorescence measurement at FRET channel. PBS with Sypro Orange alone was used as negative control and showed no fluorescence signal.

Binding kinetics. Binding kinetics of TXP1, TXP1D1 and TXP1D4 were determined by surface plasmon resonance (SPR) using a Biacore T200 (GE Healthcare). A His-capture kit (GE Healthcare) was used to immobilize anti-His antibodies on CM5 chips (as recommended by the manufacturer). His-tagged recombinant hTfR1 in 0.1% BSA in HBS-EP+ buffer (GE Healthcare) was captured at flow rate 10 μl/min. Analyte binding was measured using the multi-cycle kinetic SPR method. Starting analyte concentrations at 400 nM and serially diluted in 1:4 in seven steps. Association and dissociation were measured for 360s and 1500s respectively, with flow rate at 30 μl/min. The chips were regenerated in 10 mM Glycine-HCl, pH 1.5. Sensorgrams were fitted using 1:1 binding model and kinetic constants determined using Biacore T200 Evaluation software (Cytiva).

Flow cytometry. hCMEC/D3 cells were seeded onto V-bottom 96-well plates at a density of 1×10⁴ cells/well. All incubations were carried out on ice to prevent internalization. After blocking in PBS containing 10% BSA (FACS buffer), cells were incubated with serial dilutions of TXP1. Cells were stained with propidium iodine (PI; Biolegend) for 20 min, followed by anti-hFc conjugated to Alexa647 diluted 1:300 (Jackson ImmunoResearch) for 30 min. After fixation in 4% PFA for 15 min, the cells were analyzed on a CytoFlex flow cytometer (Beckman Coulter). Gating of cells stained with secondary antibody alone was used to set the negative threshold for and the fluorescence intensity of approximately 1000 cells per condition was measured. EC50 values were calculated using a four parametric non-linear regression model.

Cell internalization. hCMEC/D3 or SH-SY5Y cells (100,000 cells per well in 150 μL) were plated onto collagen coated 16-well chamber slides 2 days before the assay. TXP1, TXP1D1 and TXP1D4 at 44.3 nM concentration were added to the cells for 1 hr at 37° C. (5% CO₂) to allow internalization. The cells were washed twice with 300 μL of PBS, then fixed in 4% PFA in PBS for 15 min and washed again 3 times with PBS. Cells were permeabilize with PBS-0.1% saponin for 10 minutes at RT and then blocked for 30 min PBS-0.1% saponin including 5% Goat Serum. Cells were incubated with goat anti-human IgG Alexa555-conjugated antibody (Thermo #21433 diluted 1:200) for 1 hr in the dark and then washed twice with PBS-0.1% TritonX-100 and incubated with Hoechst 33342 (Thermo #H3570 diluted 1:10,000) for 10 min at RT. Finally, the cells were washed twice with PBS and examined at 20× and 60× magnification using a fluorescent microscope.

TABLE 5
Deimmunizing Mutations in Region 1,
Region 2 or Region 3 and Combinations

| Name | Region | Mutation | ** |
|---|---|---|---|
| TXP1-R1-1 | 1 | I10A | P |
| TXP1-R1-2 | 1 | I10E | A |
| TXP1-R1-3 | 1 | I10S | A |
| TXP1-R1-4 | 1 | I10T | A |
| TXP1-R1-5 | 1 | E13R, G15N, S17G | I |
| TXP1-R1-6 | 1 | T14_G15insG | I |
| TXP1-R2-1 | 2 | Y55D | I |
| TXP1-R2-2 | 2 | Y55H | A |
| TXP1-R2-3 | 2 | Y55N | A |
| TXP1-R2-4 | 2 | Y55S | A |
| TXP1-R2-5 | 2 | Y55T | A |
| TXP1-R2-6 | 2 | N60D, S63T | P |
| TXP1-R2-7 | 2 | R54K, Y55Q | I |
| TXP1-R2-8 | 2 | R54K, Y55E | I |
| TXP1-R2-9 | 2 | R54K, N60E | P |
| TXP1-R2-10 | 2 | R54K, N60Q | A |
| TXP1-R3-1 | 3 | Y97S | P |
| TXP1-R3-2 | 3 | G99D | P |
| TXP1-R3-3 | 3 | A102V | P |
| TXP1-R3-4 | 3 | Y97S, G99D, A102V | P |
| TXP1D1 | 1, 2, 3 | I10A, N60D, S63T, Y97S, G99D, A102V | P |
| TXP1D2 | 1, 2, 3 | I10A, R54K, Y55Q, Y97S, G99D, A102V | I |
| TXP1D3 | 1, 2, 3 | I10A, R54K, Y55E, Y97S, G99D, A102V | I |
| TXP1D4 | 1, 2, 3 | I10A, R54K, N60E, Y97S, G99D, A102V | P |
| TXP1D5 | 1, 2, 3 | I10A, R54K, N60Q, Y97S, G99D, A102V | A |
| TXP1D6 | 1, 2, 3 | E13R, G15N, S17G, N60D, S63T, Y97S, | I |
| | | G99D, A102V | |
| TXP1D7 | 1, 2, 3 | E13R, G15N, S17G, R54K, Y55Q, Y97S, | I |
| | | G99D, A102V | |
| TXP1D8 | 1, 2, 3 | E13R, G15N, S17G, R54K, Y55E, Y97S, | I |
| | | G99D, A102V | |
| TXP1D9 | 1, 2, 3 | E13R, G15N, S17G, R54K, N60E, Y97S, | I |
| | | G99D, A102V | |
| TXP1D10 | 1, 2, 3 | E13R, G15N, S17G, R54K, N60Q, Y97S, | I |
| | | G99D, A102V | |
| TXP1D11 | 1, 2, 3 | T14_G15insG, N60D, S63T, Y97S, G99D, | I |
| | | A102V | |
| TXP1D12 | 1, 2, 3 | T14_G15insG, R54K, Y55Q, Y97S, G99D, | I |
| | | A102V | |
| TXP1D13 | 1, 2, 3 | T14_G15insG, R54K, Y55E, Y97S, G99D, | I |
| | | A102V | |
| TXP1D14 | 1, 2, 3 | T14_G15insG, R54K, N60E, Y97S, G99D, | I |
| | | A102V | |
| TXP1D15 | 1, 2, 3 | T14_G15insG, R54K, N60Q, Y97S, G99D, | I |
| | | A102V | |
| TXP1D16 | 1, 2, 3 | I10A, Y55D, Y97S, G99D, A102V | I |
| TXP1D17 | 1, 2, 3 | I10A, Y55H, Y97S, G99D, A102V | A |
| TXP1D18 | 1, 2, 3 | I10A, Y55N, Y97S, G99D, A102V | A |
| TXP1D19 | 1, 2, 3 | I10A, Y55S, Y97S, G99D, A102V | A |
| TXP1D20 | 1, 2, 3 | I10A, Y55T, Y97S, G99D, A102V | A |
| TXP1D21 | 1, 2, 3 | I10E, Y55D, Y97S, G99D, A102V | I |
| TXP1D22 | 1, 2, 3 | I10E, Y55H, Y97S, G99D, A102V | I |
| TXP1D23 | 1, 2, 3 | I10E, Y55N, Y97S, G99D, A102V | I |
| TXP1D24 | 1, 2, 3 | I10E, Y55S, Y97S, G99D, A102V | I |
| TXP1D25 | 1, 2, 3 | I10E, Y55T, Y97S, G99D, A102V | I |
| TXP1D26 | 1, 2, 3 | I10S, Y55D, Y97S, G99D, A102V | I |
| TXP1D27 | 1, 2, 3 | I10S, Y55H, Y97S, G99D, A102V | A |
| TXP1D28 | 1, 2, 3 | I10S, Y55N, Y97S, G99D, A102V | I |
| TXP1D29 | 1, 2, 3 | I10S, Y55S, Y97S, G99D, A102V | I |
| TXP1D30 | 1, 2, 3 | I10S, Y55T, Y97S, G99D, A102V | I |
| TXP1D31 | 1, 2, 3 | I10T, Y55D, Y97S, G99D, A102V | I |
| TXP1D32 | 1, 2, 3 | I10T, Y55H, Y97S, G99D, A102V | A |
| TXP1D33 | 1, 2, 3 | I10T, Y55N, Y97S, G99D, A102V | I |
| TXP1D34 | 1, 2, 3 | I10T, Y55S, Y97S, G99D, A102V | I |
| TXP1D35 | 1, 2, 3 | I10T, Y55T, Y97S, G99D, A102V | I |

** Based on the level of expression and TfR1 binding, mutations categorized as preferred (P), active (A) or inactive (I).

TABLE 6
Comparative Properties of TXP1, TXP1D1 and TXP1D4.

| Parameter | TXP1 | TXP1D1 | TXP1D4 |
|---|---|---|---|
| Expression yield [mg/l] | 49 | 78 | 65 |
| Expression quality | 92.3 | 82.4 | 65.7 |
| (monomer peak) [%] | | | |
| Purification yields | 24 | 38.5 | 25 |
| (>97% purity) [mg/l] | | | |
| Tm by thermal shift assay [° C.] | 61.14 | 60.92 | 60.82 |
| hTfR1 binding EC50 by ELISA [M] | 1.9E−09 | 3.5E−09 | 4.1E−09 |
| cTfR1 binding EC50 by ELISA [M] | 1.4E−09 | 2.2E−09 | 3.0E−09 |
| hTfR1 affinity KD by SPR [M] | 5.1E−09 | 4.6E−09 | 3.0E−09 |
| hTfR1 on-rate by SPR [1/Ms] | 5.4E+04 | 6.7E+04 | 8.7E+04 |
| hTfR1 off-rate by SPR [1/s] | 2.7E−04 | 3.1E−04 | 2.6E−04 |
| cTfR1 affinity KD by SPR [M] | 1.8E−09 | 2.2E−09 | 1.7E−09 |
| cTfR1 on-rate by SPR [1/Ms] | 5.3E+04 | 7.3E+04 | 8.7E+04 |
| cTfR1 off-rate by SPR [1/s] | 9.6E−05 | 1.6E−04 | 1.4E−04 |
| hCMEC/D3 cell binding EC50 [M] | 2.2E−09 | 1.6E−09 | 2.1E−09 |

TABLE 7
Sequences

| SEQ ID | |
|---|---|
| NO: | SEQUENCE LISTING |
| 1 | ITKETGESL |
| 2 | YVETVNSGSK |
| 3 | YGGGTAVTVN |
| 4 | ARVDQTPQTITKETGESLTINCVLR |
| 5 | TYWYRKKSGSTNEENISKGGRYVETVNSGSKSFSLKINDLTVEDSGTYRCN |
| 6 | YGGGTAVTVNA |
| 7 | ARVDQTPQTITKETGESLTINCVLRDSNCALSSTYWYRKKSGSTNEENISKGGR |
| | YVETVNSGSKSFSLKINDLTVEDSGTYRCNVVGTWCMSWRDVYGGGTAVTVNA |
| 8 | DSNCALSS |
| 9 | TNEENISKG |
| 10 | 1ARVDQTPQTITKETGESLTINCVLR-CDR1-³⁴TYWYRKKSGSTNEENISKG |
| | GRYVETVNSGSKSFSLKINDLTVEDSGTYRCN-CDR3-⁹⁷YGGGTAVTVNA |
| 11 | SGSKS |
| 12 | VVGTWCMSWRDV |
| 13 | TYWYRKKSGSTNEENISKGGRYVETVDSGTKSFSLKINDLTVEDSGTYRCN |
| 14 | TYWYRKKSGSTNEENISKGGRKYVETVESGSKSFSLKINDLTVEDSGTYRCN |
| 15 | ARVDQTPQTATKETGESLTINCVLR-CDR1-TYWYRKKSGSTNEENISK |
| | GGRYVETVDSGTKSFSLKINDLTVEDSGTYRCN-CDR3-SGDGTVVTVNA |
| 16 | ARVDQTPQTATKETGESLTINCVLRDSNCALSSTYWYRKKSGSTNEENISKGGR |
| | YVETVDSGTKSFSLKINDLTVEDSGTYRCNVVGTWCMSWRDVSGDGTVVTVNA |
| 17 | ARVDQTPQTATKETGESLTINCVLR-CDR1-TYWYRKKSGSTNEENISK |
| | GGRKYVETVESGSKSFSLKINDLTVEDSGTYRCN-CDR3-SGDGTVVTVNA |
| 18 | ARVDQTPQTATKETGESLTINCVLRDSNCALSSTYWYRKKSGSTNEENISKGGR |
| | KYVETVESGSKSFSLKINDLTVEDSGTYRCNVVGTWCMSWRDVSGDGTVVTVNA |
| 19 | ARVDQTPQTATKETGESLTINCVLR |
| 20 | SGDGTVVTVNA |

### REFERENCES

Boado et al (2009) “Engineering and Expression of a Chimeric Transferrin Receptor Monoclonal Antibody for Blood-Brain Barrier Delivery in the Mouse,” Biotechnol. Bioeng. 102:1251-8.
Daniels et al. (2006) “The transferrin receptor part I: Biology and targeting with cytotoxic antibodies for the treatment of cancer.” Clin. Immunol. 121:144-58.
Demogines et al. (2013). “Dual host virus arms races shape an essential housekeeping protein.” PLOS Biol. 11(5):e1001571.
Forejtnikovà et al. (2010) “Transferrin receptor 2 is a component of the erythropoietin receptor complex and is required for efficient erythropoiesis,” Blood 116:5357-67.
Gerhardt et al. (1991) “The cDNA sequence and primary structure of the chicken transferrin receptor.” Gene 102:249-54.
Kariolis et al. (2020) “Brain delivery of therapeutic proteins using an Fc fragment blood-brain barrier transport vehicle in mice and monkeys.” Sci. Transl. Med. 12:eaay 1359.
Könning et al. (2017) “Camelid and shark single antibodies: structural features and therapeutic potential.” Curr. Opin. Struct. Biol. 45:10-16.
Lo et al. (2017) “Effector-attenuating Substitutions That Maintain Antibody Stability and Reduce Toxicity in Mice.” J. Biol. Chem. 292:3900-3908.
Niewoehner et al. (2014) “Increased Brain Penetration and Potency of a Therapeutic Antibody Using a Monovalent Molecular Shuttle,” Neuron 81:49-60.
Pardridge et al. (2018) “Blood-Brain Barrier Transport, Plasma Pharmacokinetics, and Neuropathology Following Chronic Treatment of the Rhesus Monkey with a Brain Penetrating Humanized Monoclonal Antibody Against the Human Transferrin Receptor.” Mol. Pharm. 15:5207-5216.
Reynisson et al. (2020) “Improved Prediction of MHC II Antigen Presentation through Integration and Motif Deconvolution of Mass Spectrometry MHC Eluted Ligand Data,” J Proteome Res. 19:2304-2315.
Silvestri et al. (2014) “The extrahepatic role of TFR2 in iron homeostasis,” Front. Pharmacol. 5:93, 6 pages.
Stocki et al. (2020) “Blood-brain barrier transport using a high-affinity, brain-selective VNAR (Variable Domain of New Antigen Receptor) antibody targeting transferrin receptor 1.” bioRxiv preprint, posted Jul. 20, 2020.
Weksler et al. (2013) “The hCMEC/D3 cell line as a model of the human blood brain barrier.” Fluids Barriers CNS 10:16.
White et al. (1990) “Combinations of anti-transferrin receptor monoclonal antibodies inhibit human tumor cell growth in vitro and in vivo: evidence for synergistic antiproliferative effects.” Cancer Res. 50:6295-301.
White et al. (1992). “Monoclonal antibodies against defined epitopes of the human transferrin receptor cytoplasmic tail.” Biochim. Biophys. Acta. 11136(1): 28-34.
Yu et al. (2014) “Therapeutic bispecific antibodies cross the blood-brain barrier in nonhuman primates,” Sci. Transl. Med. 3:84ra44, 10 pages.
Könning et al. (2017) “Camelid and shark single antibodies: structural features and therapeutic potential.” Curr. Opin. Struct. Biol. 45:10-16.
Safdari et al. (2013) “Antibody humanization methods—a review and update.” Biotechnol. Genet. Eng. Rev. 29:175-86
Kovalenko et al. (2013) “Atypical antigen recognition mode of a shark immunoglobulin new antigen receptor (IgNAR) variable domain characterized by humanization and structural analysis.” J. Biol. Chem. 288(24): 17408-19.
De Groot et al. (2005) ‘De-immunization of therapeutic proteins by T-cell epitope modification.” Dev. Biol. (Basel) 122:171-94.
Reynisson et al. (2020) “Improved Prediction of MHC II Antigen Presentation through Integration and Motif Deconvolution of Mass Spectrometry MHC Eluted Ligand Data.” J. Proteome Res. 19(6): 2304-2315.
Crooks et al. (2004) “WebLogo: a sequence logo generator.” Genome Res. 14(6): 1188-1190.
Stanfield et al. (2007) “Maturation of shark single-domain (IgNAR) antibodies: evidence for induced-fit binding.” J. Mol. Biol. 367(2): 358-72.
Henikoff et al. (1992) “Amino acid substitution matrices from protein blocks.” Proc. Natl. Acad. Sci. USA 89(22): 10915-9.
Stocki et al. (2021) “Blood-brain barrier transport using a high affinity, brain-selective VNAR antibody targeting transferrin receptor 1.” FASEB J. 35(2): e21172.

1. (canceled)

2. The VNAR domain of claim 34, wherein said amino acid changes are only made in region 1, and said change is I10A, I10E, I10S, or I10T.

3. The VNAR domain of claim 34, wherein said amino acid changes are only made in region 2, and said change is (a) a one amino acid change of Y55H, Y55N, Y55S or Y55T, or (b) a two amino acid change of N60D and S63T, R54K and N60E, or R54K and N60Q.

4. The VNAR domain of claim 34, wherein said amino acid changes are only made in region 3, and said change is (a) a one amino acid change of Y97S or A102V, (b) a two amino acid change of any two of Y97S, G99D or A102V or (c) a three amino acid change of Y97S, G99D and A102V.

5. The VNAR domain of claim 34, wherein the amino acid sequence between CDR1 and CDR3 is
(SEQ ID NO: 13)
TYWYRKKSGSTNEENISKGGRYVETVDSGTKSFSLKINDL
TVEDSGTYRCN
or
(SEQ ID NO: 14)
TYWYRKKSGSTNEENISKGGRKYVETVESGSKSFSLKIN
DLTVEDSGTYRCN

6. The VNAR domain of claim 34, wherein at least one amino acid is changed in each of region 1, 2 and 3.

7. The VNAR domain of claim 6, wherein said amino acid changes are selected from the group consisting of
I10A, N60D, S63T, Y97S, G99D and A102V;
I10A, R54K, N60E, Y97S, G99D and A102V;
I10A, R54K, N60Q, Y97S, G99D and A102V;
I10A, Y55H, Y97S, G99D and A102V;
I10A, Y55N, Y97S, G99D and A102V;
I10A, Y55S, Y97S, G99D and A102V;
I10A, Y55T, Y97S, G99D and A102V;
I10S, Y55H, Y97S, G99D and A102V; and
I10T, Y55H, Y97S, G99D and A102V.

8. The VNAR domain of claim 34, wherein said target of interest is a B-lymphocyte stimulator (BAFF), a transferrin receptor (TfR), CD98, another BBB-shuttling VNAR domain, or a COV2 spike protein.

9. The VNAR domain of claim 34, which further comprises at least one heterologous agent to thereby form a conjugate.

10. The VNAR domain of claim 9, wherein said agent is selected from the group consisting of one or more of a small molecule, a DNA, RNA, or hybrid DNA-RNA, a traceable marker such as a fluorescent or phosphorescent molecule, a radionuclide or other radioactive agent, an antibody, single chain variable domain, immunoglobulin fragment, variant or fusion, a small molecule diagnostic or therapeutic.

11. The VNAR domain of claim 10, wherein said agent is an immunoglobulin fragment.

12. The VNAR domain of claim 11, wherein said immunoglobulin fragment is an Fc domain.

13.-19. (canceled)

20. The VNAR domain of claim 34, wherein said VNAR domain comprises a CDR1 region having an amino acid sequence of DSNCALSS (SEQ ID NO: 8).

21. The VNAR domain of claim 34, wherein said VNAR domain comprises a CDR3 region having an amino acid comprising from 7 to 25 amino acids.

22. The VNAR domain of claim 34, wherein the VNAR domain comprises SEQ ID NO: 15 or 17.

23. A nucleic acid encoding the VNAR domain of claim 34 or a conjugate thereof.

24. A vector comprising a nucleic acid of claim 23.

25. A host cell comprising the vector of claim 24.

26. A method of deimmunizing a VNAR scaffold which comprises introducing one or more a deimmunizing mutations at an MHC Class II binding site in regions 1, 2 or 3 or any combination thereof in a naturally occurring or artificial VNAR scaffold comprising one or more of said regions,
wherein
(i) region 1 is represented by amino acid residues ITKETGESL (SEQ ID NO: 1),
(ii) (ii) region 2 is represented by amino acid residues YVETVNSGSK (SEQ ID NO: 2) and
(iii) (iii) region 3 is represented by amino acid residues YGGGTAVTVN (SEQ ID NO: 3).

27. The method of claim 26, wherein the Type II VNAR scaffold is for a VNAR domain having a formula of, from N to C terminus of
FW1-CDR1-FW2-HV2-FW2′-HV4-FW3-CDR3-FW4
and said scaffold comprises an amino acid sequence wherein
FW1 is
(SEQ ID NO: 4)
ARVDQTPQTITKETGESLTINCVLR;
HV2--FW2'--HV4--FW3 is
(SEQ ID NO: 5)
TYWYRKKSGSTNEENISKGGRYVETVNSGSKSFSLKIN
DLTVEDSGTYRCN;
and
FW4 is
(SEQ ID NO: 6)
YGGGTAVTVNA.

28.-33. (canceled)

34. A Type II VNAR domain having a formula of, from N to C terminus,
FW1-CDR1-FW2-HV2-FW2′-HV4-FW3-CDR3-FW4
and comprising one or more of regions 1, 2 or 3,
wherein CDR1 and CDR3 are a Type II cognate pair for a target antigen of interest;
wherein
(i) region 1 is represented by amino acid residues ITKETGESL (SEQ ID NO: 1),
(ii) region 2 is represented by amino acid residues YVETVNSGSK (SEQ ID NO: 2), and
(iii) region 3 is represented by amino acid residues YGGGTAVTVN (SEQ ID NO: 3);
wherein at least one of regions 1, 2 or 3 has one, two or three amino acids changes relative to the representative amino acid residues and which changes reduce or remove the immunogenicity of an MHC class II binding site; and
wherein said amino acid changes comprise at least one of the following mutations, taken independently or in combination,
(i) in region 1, a change of I10A, 110E, 110S, or I10T,
(ii) in region 2, (a) a one amino acid change of Y55H, Y55N, Y55S or Y55T, or (b) a two amino acid change of N60D and S63T, R54K and N60E, or R54K and N60Q,
(iii) in region 3, (a) a one amino acid change of Y97S or A102V, (b) a two amino acid change of any two of Y97S, G99D or A102V or (c) a three amino acid change of Y97S, G99D and A102V; or
a change in which at least one amino acid is changed in each of region 1, 2 and 3, wherein any of the foregoing are included as well as amino acid changes selected from the group consisting of I10A, N60D, S63T, Y97S, G99D and A102V; I10A, R54K, N60E, Y97S, G99D and A102V; I10A, R54K, N60Q, Y97S, G99D and A102V; I10A, Y55H, Y97S, G99D and A102V; I10A, Y55N, Y97S, G99D and A102V; 110A, Y55S, Y97S, G99D and A102V; 110A, Y55T, Y97S, G99D and A102V; I10S, Y55H, Y97S, G99D and A102V; and I10T, Y55H, Y97S, G99D and A102V.

35. The VNAR domain of claim 5, wherein FW1 is
(SEQ ID NO: 19)
ARVDQTPQTATKETGESLTINCVLR.

36. The VNAR domain of claim 5, wherein FW4 is SGDGTVVTVNA (SEQ ID NO: 20).


SEQUENCE LISTING (20 sequences)

SEQ ID NO: 1 - protein, 9 aa, Synthetic construct
ITKETGESL

SEQ ID NO: 2 - protein, 10 aa, synthetic construct
YVETVNSGSK

SEQ ID NO: 3 - protein, 10 aa, synthetic construct
YGGGTAVTVN

SEQ ID NO: 4 - protein, 25 aa, synthetic construct
ARVDQTPQTITKETGESLTINCVLR

SEQ ID NO: 5 - protein, 51 aa, synthetic construct
TYWYRKKSGSTNEENISKGGRYVETVNSGSKSFSLKINDLTVEDSGTYRCN

SEQ ID NO: 6 - protein, 11 aa, synthetic construct
YGGGTAVTVNA

SEQ ID NO: 7 - protein, 107 aa, synthetic construct
ARVDQTPQTITKETGESLTINCVLRDSNCALSSTYWYRKKSGSTNEENISKGGRYVETVNSGSKSFSLKINDLTVEDSGTYRCNVVGTWCMSWRDVYGGGTAVTVNA

SEQ ID NO: 8 - protein, 8 aa, synthetic construct
DSNCALSS

SEQ ID NO: 9 - protein, 9 aa, synthetic construct
TNEENISKG

SEQ ID NO: 10 - protein, 89 aa, synthetic construct
  VAR_SEQ 26: note = X=CDR1 of a shark VNAR, typically eight to 10 amino acids
  VAR_SEQ 78: note = X=CDR3 of a shark VNAR, typically eight to 25 amino acids
ARVDQTPQTITKETGESLTINCVLRXTYWYRKKSGSTNEENISKGGRYVETVNSGSKSFSLKINDLTVEDSGTYRCNXYGGGTAVTVNA

SEQ ID NO: 11 - protein, 5 aa, synthetic construct
SGSKS

SEQ ID NO: 12 - protein, 12 aa, synthetic construct
VVGTWCMSWRDV

SEQ ID NO: 13 - protein, 51 aa, synthetic construct
TYWYRKKSGSTNEENISKGGRYVETVDSGTKSFSLKINDLTVEDSGTYRCN

SEQ ID NO: 14 - protein, 52 aa, synthetic construct
TYWYRKKSGSTNEENISKGGRKYVETVESGSKSFSLKINDLTVEDSGTYRCN

SEQ ID NO: 15 - protein, 89 aa, synthetic construct
  VAR_SEQ 26: note = X=CDR1 of a shark VNAR, typically eight to 10 amino acids
  VAR_SEQ 78: note = X=CDR3 of a shark VNAR, typically eight to 25 amino acids
ARVDQTPQTATKETGESLTINCVLRXTYWYRKKSGSTNEENISKGGRYVETVDSGTKSFSLKINDLTVEDSGTYRCNXSGDGTVVTVNA

SEQ ID NO: 16 - protein, 107 aa, synthetic construct
ARVDQTPQTATKETGESLTINCVLRDSNCALSSTYWYRKKSGSTNEENISKGGRYVETVDSGTKSFSLKINDLTVEDSGTYRCNVVGTWCMSWRDVSGDGTVVTVNA

SEQ ID NO: 17 - protein, 90 aa, synthetic construct
  VAR_SEQ 26: note = X=CDR1 of a shark VNAR, typically eight to 10 amino acids
  VAR_SEQ 79: note = X=CDR3 of a shark VNAR, typically eight to 25 amino acids
ARVDQTPQTATKETGESLTINCVLRXTYWYRKKSGSTNEENISKGGRKYVETVESGSKSFSLKINDLTVEDSGTYRCNXSGDGTVVTVNA

SEQ ID NO: 18 - protein, 108 aa, synthetic construct
ARVDQTPQTATKETGESLTINCVLRDSNCALSSTYWYRKKSGSTNEENISKGGRKYVETVESGSKSFSLKINDLTVEDSGTYRCNVVGTWCMSWRDVSGDGTVVTVNA

SEQ ID NO: 19 - protein, 25 aa, synthetic construct
ARVDQTPQTATKETGESLTINCVLR

SEQ ID NO: 20 - protein, 11 aa, synthetic construct
SGDGTVVTVNA